SEQUENCE LISTING (16 sequences)

SEQ ID NO: 1 - protein, 27 aa, Artificial Sequence
  CMAP27
RFGRFLRKIRRFRPKVTITIQGSARFG

SEQ ID NO: 2 - protein, 21 aa, Artificial Sequence
  cycCMAP(1-21)[Lys8]
  MISC_FEATURE (8)..(8): is Fmoc-L-bishomopropargylglycine
  MISC_FEATURE (21)..(21): azido resin attached
RFGRFLRXIRRFRPKVTITIQ

SEQ ID NO: 3 - protein, 21 aa, Artificial Sequence
  cycCMAP(1-21)[Arg7]
  MISC_FEATURE (7)..(7): is Fmoc-L-bishomopropargylglycine
  MISC_FEATURE (21)..(21): azido resin attached
RFGRFLXKIRRFRPKVTITIQ

SEQ ID NO: 4 - protein, 21 aa, Artificial Sequence
  cycCMAP(1-21)[Leu6]
  MISC_FEATURE (6)..(6): is Fmoc-L-bishomopropargylglycine
  MISC_FEATURE (21)..(21): azido resin attached
RFGRFXRKIRRFRPKVTITIQ

SEQ ID NO: 5 - protein, 21 aa, Artificial Sequence
  cycCMAP(1-21)[Leu6],Phe2/Trp
  MISC_FEATURE (6)..(6): is Fmoc-L-bishomopropargylglycine
  MISC_FEATURE (21)..(21): azido resin attached
RWGRFXRKIRRFRPKVTITIQ

SEQ ID NO: 6 - protein, 21 aa, Artificial Sequence
  cycCMAP(1-21)[Leu6],Phe2,5/Trp
  MISC_FEATURE (6)..(6): is Fmoc-L-bishomopropargylglycine
  MISC_FEATURE (21)..(21): azido resin attached
RWGRWXRKIRRFRPKVTITIQ

SEQ ID NO: 7 - protein, 21 aa, Artificial Sequence
  cycCMAP(1-21)[Leu6],Phe2,5,12/Trp
  MISC_FEATURE (6)..(6): is Fmoc-L-bishomopropargylglycine
  MISC_FEATURE (21)..(21): azido resin attached
RWGRWXRKIRRWRPKVTITIQ

SEQ ID NO: 8 - protein, 21 aa, Artificial Sequence
  cycCMAP(1-21)[Leu6],Phe5,12/Trp
  MISC_FEATURE (6)..(6): is Fmoc-L-bishomopropargylglycine
  MISC_FEATURE (21)..(21): azido resin attached
RFGRWXRKIRRWRPKVTITIQ

SEQ ID NO: 9 - protein, 21 aa, Artificial Sequence
  cycCMAP(1-21)[Leu6],Phe12/Trp
  MISC_FEATURE (6)..(6): is Fmoc-L-bishomopropargylglycine
  MISC_FEATURE (21)..(21): azido resin attached
RFGRFXRKIRRWRPKVTITIQ

SEQ ID NO: 10 - protein, 27 aa, Artificial Sequence
  RI/I Cath2
GFRASGQITITVKPRFRRIKRLFRGFR

SEQ ID NO: 11 - protein, 21 aa, Artificial Sequence
  RI-C(1-21)
QITITVKPRFRRIKRLFRGFR

SEQ ID NO: 12 - protein, 18 aa, Artificial Sequence
  RI-C(4-21)
QITITVKPRFRRIKRLFR

SEQ ID NO: 13 - protein, 15 aa, Artificial Sequence
  RI-C(7-21)
QITITVKPRFRRIKR

SEQ ID NO: 14 - protein, 15 aa, Artificial Sequence
  RI-C(7-21)F/W
QITITVKPRWRRIKR

SEQ ID NO: 15 - protein, 15 aa, Artificial Sequence
  RI-C(7-21)F/Y
QITITVKPRYRRIKR

SEQ ID NO: 16 - protein, 12 aa, Artificial Sequence
  RI-C(10-21)F/W
QITITVKPRWRR

(19) United States
(12) Patent Application Publication
(10) Pub. No.: US 2024/0165200 A1
(43) Pub. Date: May 23, 2024

(54) CATH2 AND DERIVATIVES FOR INHIBITING STREPTOCOCCUS SUIS

(71) Applicant: Universiteit Utrecht Holding B.V., Utrecht (NL)

(72) Inventors: Maaike Riena SCHEENSTRA, Leiderdorp (NL); Roeland Maarten VAN HARTEN, Utrecht (NL); Hendrik Peter HAAGSMAN, Zeist (NL); Albert VAN DIJK, Zeist (NL); Edwin Johannes Adrianus VELDHUIZEN, Utrecht (NL)

(21) Appl. No.: 18/275,155

(22) PCT Filed: Feb. 4, 2022
(86) PCT No.: PCT/NL2022/050057

§ 371 Date: Jul. 31, 2023

(30) Foreign Application Priority Data
Feb. 5, 2021 (EP) 21155491.0

(51) Int. Cl. A61K 38/17; A61K 39/00; A61K 39/39; A61P 31/04

(52) U.S. Cl. CPC A61K 38/1729; A61K 39/39; A61P 31/04; A61K 2039/5555; A61K 2039/55561; A61K 2039/55583

(57) ABSTRACT

The invention relates to methods for inhibiting S. suis comprising administering to CATH2 or a derivative thereof and to methods for the treatment or prevention of a S. suis infection in a subject in need thereof, comprising administering CATH2 or a derivative thereof to the subject.

### FIELD OF THE INVENTION

The invention relates to the field of medical and veterinary science, in particular to use of CATH2 derivatives in infectious disease.

### BACKGROUND OF THE INVENTION

Streptococcus suis (S. suis) is a Gram-positive facultative anaerobe bacterium, with a spherical shape and contains alpha hemolysis on agar plates containing blood.[1,2] S. suis is found in almost all pigs as a commensal part of the respiratory microbiota, but can also cause invasive infections, such as meningitis, endocarditis, and sudden death. [2] In addition, S. suis is an emerging zoonotic agent and can cause sepsis and meningitis in human.[3] It contains a large polysaccharide capsule to prevent phagocytosis-dependent clearing.[4] Up to 35 various serotypes of S. suis have been identified so far, of which serotype 2 is found most often in diseased pigs, followed by serotype 9 and 3. In human cases serotype 2 is found most often.[5] Various antibiotics have been used for treating diseases caused by S. suis. However, due to expansion of antibiotic resistant S. suis strains, the development of novel therapeutics are urgently needed.

Cathelicidins are host defense peptides (HDPs) and part of the innate immune system [8]. These peptides are known endogenous alarmins that are passively (necrosis) or actively released through microbial exposure or neutrophil and mast cell degranulation upon tissue injury or infection [9]. Potent immunomodulatory effects on macrophages have been reported for human cathelicidin LL-37 and chicken CATH2 in vitro [10-13]. To increase the therapeutic potential of cathelicidin-derived peptides, a full D-amino acid analog can be used to gain high resistance against proteases while maintaining low immunogenicity [14]. Prophylactic treatment of chicken embryos by in ovo injection with DCATH2 considerably reduced colibacilossis-associated mortality and morbidity [15]. In addition, delayed mortality was observed when DCATH2 was injected into the yolk of zebrafish embryos followed by intravenously infection with a lethal dose of Salmonella enterica [16].

Although several antibiotic and antimicrobial treatments are known, there remains a need in the art for improved methods of treatment and prevention of in particular infectious disease, including S. suis.

### SUMMARY OF THE INVENTION

It is an object of the present invention to provide novel uses of CATH2 and derivatives thereof. It is a further object of the invention to provide effective inhibition, treatment and/or prevention of S. suis and S. suis infection. The invention therefore provides a method for the treatment and/or prevention of a S. suis infection in a subject in need thereof comprising administering to the subject CATH2 or a derivative thereof.

In a further aspect, the invention provides CATH2 or a derivative thereof for use in a method for the treatment and/or prevention of a S. suis infection in a subject in need thereof.

In a further aspect, the invention provides a use of CATH2 or a derivative thereof in the preparation of a medicament for the treatment and/or prevention of a S. suis infection in a subject in need thereof.

In a further aspect, the invention provides a method for inhibiting Streptococcus suis comprising administering CATH2 or a derivative thereof to the S. suis.

In a further aspect, the invention provides CATH2 or a derivative thereof for use in a method for inhibiting S. suis.

In a further aspect, the invention provides a use of CATH2 or a derivative thereof in the preparation of a medicament for inhibiting Streptococcus suis.

In one embodiment, the S. suis is preferably S. suis serotype 2, serotype 9, serotype 1 or serotype 3, preferably serotype 2.

In one embodiment, the subject is preferably a mammal or an avian subject, preferably a human, a bovine, including dairy and beef cattle, other ruminant including a sheep, a pig, poultry, a dog, a cat or a horse.

The subject in need is preferably suffering from a S. suis infection or at risk of suffering from a S. suis infection, such as a subject that is in contact with subjects suffering from said infection.

In one embodiment, the methods and uses of the invention further comprise inducing or promoting innate immune memory in the subject. In another embodiment, the methods and uses of the invention further comprise improving or enhancing antimicrobial treatment with an antimicrobial agent and/or antimicrobial activity of an antimicrobial agent.

In one embodiment, the CATH2 derivative is selected from the group consisting of DCATH2, a C-terminally and/or N-terminally truncated CATH2 and a C-terminally or N-terminally truncated DCATH2, preferably selected from the group consisting of DCATH2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

In one embodiment, the CATH2 or derivative is DCATH2, DCATH2(1-21) or DCATH2(4-21).

### DETAILED DESCRIPTION

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 10% of the value.

In one embodiment, the methods and uses of the invention are for inhibiting S. suis. As used herein “inhibiting S. suis” refers to prevent, retard, slow, hinder, reverse, or delay growth of S. suis. In particular, “inhibit S. suis” means that growth of S. suis in the presence of the CATH2 or derivative thereof as described herein is slower than the growth of S. suis in the absence thereof. In one aspect, the growth of S. suis is slowed in the presence of the CATH2 or derivative thereof as described herein, In another aspect, S. suis growth is halted, which means that no additional growth of S. suis is observed after the addition or administration of CATH2 or derivative thereof as described herein. In another aspect, the growth of S. suis is reversed, which means that existing S. suis are killed in the presence of the CATH2 or derivative thereof as described herein. S. suis growth can for instance be determined in the presence and absence of the CATH2 or derivative thereof as described herein in an assay as described in the examples herein below.

In one embodiment, the methods and uses of the invention are for the treatment of existing disease, in particular S. suis infection. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In one embodiment, the methods and uses of the invention are for prevention of disease, in particular S. suis infection. As used herein, the term “prevention” refers to precluding or delaying the onset of a disease or condition and/or the appearance of clinical symptoms of the disease or condition in a subject that does not yet experience clinical symptoms of the disease.

The term “peptide” as used herein means a sequence of amino acids that are coupled by peptide bonds, wherein the amino acids are one of the twenty naturally peptide-building amino acids and wherein one or all of the amino acids can be in the L-configuration or in the D-configuration, or, for isoleucine and threonine in the D-allo configuration (only inversion at one of the chiral centers). A peptide according to the invention can be linear, i.e. wherein the first and last amino acids of the sequence have a free NH2- or COOH-group respectively or are N-terminally (acetylation) and/or C-terminally (amidation) modified. In amino acid sequences as defined herein amino acids are denoted by single-letter symbols or three-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

The present inventors identified CATH2 and derivatives thereof as potent inhibitors of S. suis. As demonstrated in the Examples herein, DCATH2 and truncated forms thereof were shown to have a strong direct antibacterial activity against four different S. suis strains in bacterial medium and even stronger in more physiological cell culture medium containing serum. In addition, D-CATH2 and its derivatives were shown to ameliorate the efficiency of mouse bone marrow-derived macrophages (BMDM) and skewed mouse bone marrow-dendritic cells (BMDCs) towards cells with a more macrophage-like phenotype. The peptides were able to directly bind LTA and inhibit LTA-induced activation of macrophages. In addition, the peptides killed S. suis in a silently fashion, unable to further activate mouse macrophages, which may prevent an excess immune reaction upon infection. Administration of DCATH2 derivative DCATH2(1-21) 24 h and 7 days before infection with S. suis, results in a small prophylactic protection of mice, with slightly reduced disease severity and reduced preliminary dead of the treated mice.

In a first aspect, the invention therefor provides a method for inhibiting Streptococcus suis (S. suis) comprising administering CATH2 or a derivative thereof to said S. suis. Also provided is CATH2 or a derivative thereof for use in a method for inhibiting S. suis.

In one embodiment S. suis is inhibited in a sample, and the method or use of the invention comprises administering the CATH2 or derivative thereof to said sample. In another embodiment, S. suis is inhibited in a cell, either in vivo, in vitro or ex vivo, and the method or use of the invention comprises administering the CATH2 or derivative thereof to said cell. In a preferred embodiment, S. suis is inhibited in a subject in need thereof. Hence, in a preferred embodiment the method or use comprises administering the CATH2 or derivative thereof to a subject in need thereof.

In one embodiment, the method or use is for treatment or prevention of a S. suis infection in a subject in need thereof. Also provided is therefore CATH2 or a derivative thereof for use in a method for the treatment and/or prevention of a S. suis infection in a subject in need thereof. The subject is preferably a mammal or an avian subject, more preferably the subject is a human, a bovine, including dairy and beef cattle, other ruminant including a sheep, a pig, poultry, a dog, a cat or a horse. The subject in need thereof is preferably a subject suffering from S. suis infection or at risk of suffering from S. suis infection. A subject at risk of suffering from infection is for instance a subject, preferably a bovine, including dairy and beef cattle, other ruminant including a sheep, a pig, poultry, a dog, a cat or a horse, that is in contact with subjects suffering from S. suis, e.g. if they are kept in the same space, land, stable, house or farm.

In one embodiment the CATH2 or derivative thereof is comprised in a vaccine, immunogenic composition, pharmaceutical composition or other therapeutic composition. Provided is therefore a vaccine, immunogenic composition, pharmaceutical or therapeutic composition comprising CATH2 or derivative thereof for use in a method for the prevention of a S. suis infection, preferably in a human, a bovine, including dairy and beef cattle, other ruminant including a sheep, a pig, poultry, a dog, a cat or a horse. As detailed herein, such vaccine or composition may comprise further constituents, including pharmaceutically acceptable carriers or excipients and one or more adjuvants.

S. suis is an important cause of meningitis, sepsis, endocarditis, arthritis, pneumonia and sudden death in pigs and it can also cause meningitis in man and may result in sepsis, pneumonia, arthritis, such as septic arthritis, and endocarditis as well. Hence, in one embodiment, a method of the invention for inhibiting S. suis comprises treating and/or preventing meningitis, sepsis, endocarditis, arthritis, pneumonia and/or sudden death that is associated with or resulting from S. suis infection, in particular in pigs. In another embodiment, a method of the invention for inhibiting S. suis comprises treating and/or preventing meningitis, sepsis, pneumonia, arthritis, such as septic arthritis, and/or endocarditis that is associated with or resulting from S. suis infection, in particular in man. The isolation of S. suis from poultry, dogs, cats, cattle and other ruminants, and horses has however also been reported. Hence, in another embodiment, a method of the invention comprises treating and/or preventing S. suis infection in a bovine, including dairy and beef cattle, other ruminant including a sheep, poultry, a dog, a cat or a horse.

The S. suis can be S. suis of any serotype. In a preferred embodiment, the S. suis is S. suis serotype 2, serotype 9, serotype 1 or serotype 3. In a particular embodiment, S. suis is S. suis serotype 2.

Inhibition of S. suis by CATH2 or a derivative thereof can for instance be determined using an assay as described in the examples herein, wherein the mean bactericidal concentration (MBC) of the CATH2 or derivative is to the particular S. suis is determined in bacterial growth medium. Hence, a skilled person is well capable of determining S. suis inhibitory activity of a CATH2 derivate as described herein.

As used herein, the terms “CATH2” and “CMAP27” are used interchangeably. Like other members of the cathelicidin family CMAP27 is encoded as a prepropeptide (154 amino acids) and after proteolytic processing, a C-terminal peptide is released that has demonstrated potent broad spectrum antimicrobial activity. The 27 amino acid sequence of this C-terminal peptide, called CMAP27 or CATH2, is RFGRFLRKIRRFRPKVTITIQGSARFG. As used herein, a “CATH2 derivative” generally refers to a peptide that is a derivative of CATH2 in that it contains at least part of the sequence of CATH2 and that has maintained at least one antimicrobial properties of CATH2, although not necessarily to the same extent. In particular, antimicrobial activity against S. suis bacteria is maintained.

As used herein, the terms “CATH2” and “CMAP27” are used interchangeably. Like other members of the cathelicidin family CMAP27 is encoded as a prepropeptide (154 amino acids) and after proteolytic processing, a C-terminal peptide is released that has demonstrated potent broad spectrum antimicrobial activity. The 27 amino acid sequence of this C-terminal peptide, called CMAP27 or CATH2, is RFGRFLRKIRRFRPKVTITIQGSARFG. As used herein, a “CATH2 derivative” generally refers to a peptide that is a derivative of CATH2 in that it contains at least part of the sequence of CATH2 and that has maintained at least one antimicrobial properties of CATH2, although not necessarily to the same extent. In particular, antimicrobial activity against Gram(−) bacteria is maintained.

In one preferred embodiment, the CATH2 derivative is selected from the group consisting of C-terminally and/or N-terminally truncated CATH2 derivatives, D-amino acid CATH2 derivatives, C-terminally or N-terminally truncated D-amino acid CATH2 derivatives, cyclic CATH2 derivatives and inverso and retroinverso CATH2 derivatives. The derivative may contain one or more amino acid substitutions, preferably 1 to 3 amino acid substitutions, more preferably 1 or 2 amino acid substitutions. Preferably, the CATH2 derivative is selected from the group consisting of C-terminally and/or N-terminally truncated CATH2 derivatives, D-amino acid CATH2 derivatives and C-terminally or N-terminally truncated D-amino acid CATH2 derivatives, such as C-terminally or N-terminally truncated DCATH2. In one preferred embodiment, CATH2 or DCATH2 is used. DCATH2 is the full length CATH2 peptide consisting of D-amino acids.

“C-terminally truncated CATH2 derivatives” refers to truncated peptides lacking one or more amino acids at the C-terminus of CATH2, preferably lacking up to 17 amino acids, more preferably up to 12 amino acids, more preferably up to 6 amino acids. Preferred examples are described in WO 2010/093245, which is incorporated herein by reference, and especially the peptides listed as CMAP26-NH₂, CMAP26, CMAP26 (P14→G), CMAP26 (P14→L), CMAP1-21, CMAP1-15, CMAP1-15 (F2→L), CMAP1-15 (F5→L), CMAP1-15 (F12→L), CMAP1-15 (3xF =L), CMAP1-15 (F2→W), CMAP1-15 (F5→W), CMAP1-15 (F12→W), CMAP1-15 (F2→W; F5→W; F12→W), CMAP1-13, CMAP1-12, CMAP1-11 and CMAP1-10 in Table 1 of said document and their acetylated and/or amidated derivatives are preferred. Herein, and in all amino acid sequence defined herein, the arrow notation indicates an amino acid substitution. For instance, F2→L indicates that the F at position 2 is replaced by L and F2, 5→W indicates that F at positions 2 and 5 is replaced by W. Further preferred are CMAP1-21 (F2→W), CMAP1-21 (F5→W), CMAP1-21 (F12→W), CMAP1-21 (F2, 5→W), CMAP1-21 (F5, 12→W), CMAP1-21 (F2, 12→W), CMAP1-21 (F2, 5, 12→W), CMAP1-21 (F2→Y), CMAP1-21 (F5→Y), CMAP1-21 (F12→Y), CMAP1-21 (F2, 5→Y), CMAP1-21 (F5, 12→Y), CMAP1-21 (F2, 12→Y), CMAP1-21 (F2, 5, 12→Y), CMAP1-21 (F2→W; F5→Y), CMAP1-21 (F2→Y; F5→W), CMAP1-21 (F5→W; F12→Y), CMAP1-21 (F5→Y; F12→W), CMAP1-21 (F2→W; F12→Y), CMAP1-21 (F2→Y; F12→W), CMAP1-21 (F2→W; F5→Y; F12→Y), CMAP1-21 (F2→Y; F5→W; F12→Y), and CMAP1-21 (F2→Y; F12→Y; F12→W). Preferred examples of C-terminally truncated CATH2 derivatives are also described in WO 2015/170984, which is incorporated herein by reference. The CMAP proteins identified above, may also be indicates as CATH2 peptides. CMAP1-21 then would be CATH2(1-21).

“N-terminally truncated CATH2 derivatives” are CATH2 derivatives that are truncated at the N-terminal amino acid (arginine) of CATH2 thus lacking one or more amino acids at the N-terminus of CATH2, preferably lacking up to 10 amino acids, more preferably up to 7 amino acids, more preferably up to 6 amino acids. Preferred are the derivatives selected from the group consisting of N-terminally truncated variants of CMAP1-21: CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

“D-amino acid CATH2 derivatives” are CATH2 derivatives as defined herein (including the above defined C- and N-terminally truncated CMAP27-derivatives) that contain at least one amino acid in the D configuration. A special category of these D-amino acid CATH2 derivatives are the peptides that are composed of only D amino acids (i.e. in which no L amino acid is present). This special category is herein defined as DCATH2. Also CATH2 itself, comprising one or more, or, alternatively, all D amino acids is comprised within this definition. Preferred D-amino acid CATH2 derivatives are DCATH2 and the following D-amino acid CATH2 derivatives (indicated as D-C, and where all amino acids are in the D-form):

| | |
|---|---|
| D-C(1-26) | RFGRFLRKIRRFRPKVTITIQGSARF-NH₂ |
| D-C(1-21) | RFGRFLRKIRRFRPKVTITIQ-NH₂ |
| D-C(4-21) | RFLRKIRRFRPKVTITIQ-NH₂ |
| D-C(7-21) | RKIRRFRPKVTITIQ-NH₂ |
| D-C(7-21)F/W | RKIRRWRPKVTITIQ-NH₂ |
| D-C(7-21)F/Y | RKIRRYRPKVTITIQ-NH₂ |
| D-C(10-21)F/W | RRWRPKVTITIQ-NH₂ |
| D-C(1-15) | RFGRFLRKIRRFRPK-OH |

Particularly preferred are DCATH2 and DCATH2 derivatives DCATH2(1-21) (also called DC(1-21)) and DCATH2(4-21) (also called DC(4-21)).

“Cyclic CATH2-derivatives” are CATH2 derivatives in which at least two non-adjacent amino acids are connected to form a ring structure. Although in principle any chemical binding construction may be used, such as replacing two non-adjacent amino acids in any of the above-mentioned CATH2 derivatives with a cysteine, where these cysteines then form an S-S bridge, a preferred binding system uses the binding between Bpg (Fmoc-L-bishomopropargylglycine) and an azido-resin, wherein the Bpg is attached to an internal arginine, leucine, phenylalanine or tryptophane residue and the azido-resin is attached to the C-terminal glutamic acid residue. Especially, such cyclic derivatives are:

| | |
|---|---|
| cycCMAP(1-21)[Lys8] | RFGRFLR(Bpg)IRRFRPK |
| | VTITIQ(azido-resin) |
| cycCMAP(1-21)[Arg7] | RFGRFL(Bpg)KIRRFRPK |
| | VTITIQ(azido-resin) |
| cycCMAP(1-21)[Leu6] | RFGRF(Bpg)RKIRRFRPK |
| | VTITIQ(azido-resin) |
| cycCMAP(1-21)[Leu6], | RWGRF(Bpg)RKIRRFRPK |
| Phe2/Trp | VTITIQ(azido-resin) |
| cycCMAP(1-21)[Leu6], | RWGRW(Bpg)RKIRRFRPK |
| Phe2,5/Trp | VTITIQ(azido-resin) |
| cycCMAP(1-21)[Leu6], | RWGRW(Bpg)RKIRRWRPK |
| Phe2,5,12/Trp | VTITIQ(azido-resin) |
| cycCMAP(1-21)[Leu6], | RFGRW(Bpg)RKIRRWRPK |
| Phe5,12/Trp | VTITIQ(azido-resin) |
| cycCMAP(1-21)[Leu6], | RFGRF(Bpg)RKIRRWRPK |
| Phe12/Trp | VTITIQ(azido-resin) |

“Inverso” and “Retroinverso” CATH2 derivatives (“I”-CATH2 and “RI”-CATH2 derivatives) are peptides that have an inverted sequence with respect to the above-mentioned CATH2 derivatives, in the sense that the amino acids are connected to each other in a reverse order. When the inverted CATH2 derivatives contain one or more D amino acids they are termed “Retroinverso” or “RI”. If the inverted derivative only contains L-amino acids it is termed “Inverso” or “I”. The I and RI equivalent of CATH2 then become GFRASGQITITVKPRFRRIKRLFRGFR and other preferred examples of such I or RI-CMAP27-derivatives are:

| | |
|---|---|
| RI-C(1-21) | QITITVKPRFRRIKRLFRGFR |
| RI-C(4-21) | QITITVKPRFRRIKRLFR |
| RI-C(7-21) | QITITVKPRFRRIKR |
| RI-C(7-21)F/W | QITITVKPRWRRIKR |
| RI-C(7-21)F/Y | QITITVKPRYRRIKR |
| RI-C(10-21)F/W | QITITVKPRWRR |

The I and RI-CMAP27 derivatives may be acetylated at their N-terminal and/or amidated at their C-terminal.

In a preferred embodiment, the CATH2 or derivative thereof used in any method or use of the invention is CATH2, DCATH2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y), more preferably wherein the CATH2 or derivative is CATH2, DCATH2, DCATH2(1-21) or DCATH2(4-21). In one embodiment, the CATH2 or derivative thereof used in any method or use of the invention is DCATH2, DCATH2(1-21) or DCATH2(4-21).

As used herein, the term “subject” encompasses humans and animals, including livestock and farm animals such as dairy cattle and beef cattle and other bovine, including cows and buffaloes, sheep, goats, alpacas, horses, mules, donkeys, camels, llamas, pigs, swine, fish, rodents, poultry, dogs, cats, chinchillas, ferrets, birds, hamsters, rabbits, mice, gerbils, rats, and guinea pigs. In one preferred embodiment, the subject is a mammal, such as mammalian farm animals, livestock or pets. In one preferred embodiment, the subject is a human, pig, a bovine, such as cattle, including dairy and beef cattle, poultry, sheep, horse, dog or cat. In one embodiment, the subject is an avian subject, preferably poultry. The term poultry includes chicken, ducks, goose, pheasants and turkeys. In a preferred embodiment, the poultry is chicken or turkey, more preferably chicken. Preferred subjects are pigs, cattle, poultry, sheep, horses, dogs and cats.

In one embodiment, the “subject in need thereof” is a subject in need of treatment or prevention of a S. suis infection. Examples of such subjects are subjects suffering from a S. suis infection, or at risk of suffering from a S. suis infection. In one embodiment, the subject at risk of suffering from a S. suis infection is a subject that is in contact with subjects suffering from said infection, for instance a subject in a population of subjects wherein a S. suis infection has been established in one or more subjects of said population. Hence, particularly useful is the application of CATH2 or a derivative thereof in farms or stables where animals are kept together, such as in farming, including poultry, bovine, cattle, pig, goat, sheep and horse farming. Infectious disease occurring in such environments may quickly spread throughout the facility. In one embodiment therefore, the subject in need of administering CATH2 or a derivative is suffering from an infectious disease or at risk of suffering from a S. suis infection, preferably S. suis serotype 2, serotype 9, serotype 1 or serotype 3, more preferably S. suis serotype 2. In one embodiment, the subject, preferably a bovine, including dairy and beef cattle, other ruminant including a sheep, a pig, poultry, a dog, a cat or a horse, at risk of suffering from a S. suis infection is a subject that is in contact with subjects, preferably of the same species, suffering from a S. suis infection. A subject, at risk of suffering from a S. suis infection, such as a human, a bovine, including dairy and beef cattle, other ruminant including a sheep, a pig, poultry, a dog, a cat or a horse, is for instance a subject that is in contact with subjects suffering from a S. suis infection, e.g. because they are kept in the same space, land, stable, house, kennel or farm. For instance, once a S. suis infection has been established in a kennel, farm or stable, treatment of non-infected subjects with CATH2 or derivative thereof in accordance with the present invention is beneficial. In one embodiment, both a S. suis infected subjects and subjects at risk of suffering from a S. suis infection, in particular because they are in contact with a S. suis infected subjects, are treated in accordance with the present invention. Hence, in one embodiment, the CATH2 or derivative thereof is administered to subjects of a population of subjects wherein a S. suis infection has been established in one or more subjects of said population, preferably to the majority or all of the subjects in said population. Said S. suis is preferably S. suis serotype 2, serotype 9, serotype 1 or serotype 3, more preferably S. suis serotype 2. Said subject is preferably a bovine, including dairy and beef cattle, other ruminant including a sheep, a pig, poultry, a dog, a cat or a horse and said population of subjects is preferably a population of the same species, e.g. a population of bovines, including dairy and beef cattle, other ruminant including sheep, pigs, poultry, dogs, cats or horses. Now that it has been found that CATH2 and derivatives inhibit S. suis, it is advantageous to treat both subjects already suffering from S. suis infection and subjects that are at risk of suffering therefrom effectively and concomitantly.

S. suis is an emerging zoonotic agent and can cause sepsis and meningitis in human. Therefore, in one preferred embodiment the subject is a human, more preferably a human suffering from S. suis infection or at risk of suffering from a S. suis infection, preferably S. suis serotype 2, serotype 9, serotype 1 or serotype 3, more preferably S. suis serotype 2.

It has been established by present inventors that CATH2 and derivatives as described herein are able to induce and/or stimulate trained immunity or innate immune memory, in particular innate immune memory for infectious disease. Now that this has been established, it has become possible to improve antimicrobial therapies using these, thereby improving treatment outcomes and/or treatment efficiency. In particular, the improvement is an improvement in treatment efficiency, such as in timing of administration of the CATH2 or derivative, dosing of the CATH2 or derivative, formulation of the CATH2 or derivative and/or administration routes, combination with other active or non-active compounds as described elsewhere in more detail. In particular, now that is known that CATH2 and derivatives stimulate innate immune memory, it has become possible to select an appropriate formulation, including appropriate auxiliaries, such as adjuvants, and/or combination with other active or non-active ingredients and administrations dosages and schemes that support the induction or promotion or innate immune memory. Hence, in one embodiment, the method or use according to the invention comprises inducing or promoting innate immune memory in the subject. Alternatively, or additionally, the method or use comprises improving or enhancing antimicrobial treatment with an antimicrobial agent and/or improving or enhancing antimicrobial activity of an antimicrobial agent. The treatment is in particular treatment or prevention of S. suis infection as defined herein. The antimicrobial agent is in particular the CATH2 or derivative thereof.

The terms “Innate immune memory” and “trained immunity” are used interchangeably and refer to the ability of innate immune cells to functionally reprogram after exogenous or endogenous insults and to respond non-specifically to a subsequent challenge after return a non-activated state. Trained immunity is orchestrated by epigenetic modifications leading to changes in gene expression and cell physiology of the innate immune cells. The innate immune memory provides a powerful tool to regulate the delicate balance of immune homeostasis, priming, training and tolerance of innate immune cells. The long-term adaptation demonstrated with trained immunity can be used to achieve long-term therapeutic benefits with a more strongly response in a range of immune-related diseases, including infectious disease, as compared to direct treatment with antimicrobial agents.

In the methods of the invention, CATH2 or derivative thereof are further advantageously combined with another agent capable of inducing or promoting innate immune memory or an adjuvant specific for innate immunity. Examples of such agents/adjuvants include toll-like receptor (TLR) ligands, β-glucan, muramyl dipeptide (MDP) or peptide comprising MDP, Bacille Calmette-Guerin (BCG), cytosine-guanine dinucleotide (CpG) containing oligodeoxynucleotide. TLR ligands are known to one of skill in the art and include triacyl and diacyl portions of lipoproteins (TLR2, TLR1, TLR6), flagellin (TLR5), double-stranded RNA (TLR3), single-stranded RNA (TLR7) and bacterial and viral (CpG) DNA (TLR9). MDP is a synthetic peptide conjugate comprising N-acetyl muramic acid and a short amino acid chain of L-alanine D-isoglutamine dipeptide. β-glucan is a naturally occurring polysaccharide found in the cell wall of yeast, bacteria and fungi. Bacille Calmette-Guérin (BCG) is the vaccine against Mycobacterium tuberculosis (TB). CpG oligodeoxynucleotides are generally present in viral/microbial DNA and are ligand for TLR9 as indicated above.

As described herein above, in one embodiment the subject that is treated in accordance with the present invention is poultry, such as chicken. Administration of the CATH2 or derivative in accordance with the methods of the invention may be achieved by in ovo administration to poultry embryos or by administration of young poultry after hatch. In the latter case, administration is preferably within one week after hatch, more preferably within 3 days after hatch. As used herein “in ovo administration” refers to administration to eggs of an avian species, preferably eggs in the fourth quarter of incubation. I.e. for chicken eggs, the administration is conducted preferably on about the fifteenth to nineteenth day of incubation, and more preferably on about the eighteenth day of incubation. For turkey eggs, the administration is conducted preferably on about the twenty-first to twenty-sixth day of incubation, and more preferably on about the twenty-fifth day of incubation. Such an administration can be conducted by any method which results in the introduction of one or more of the CATH2 or derivatives into an egg through the shell. A preferred method of administration is by injection. The injection can be performed by using any one of the well-known egg injection devices, such as a conventional hypodermic syringe fitted with a needle of about 18 to 22 gauge, or a high speed automated egg injection system as described in U.S. Pat. Nos. 4,681,063, 4,040,388, 4,469,047, and 4,593,646.

In one embodiment, the subject is administered the CATH2 derivative twice. The two administration are preferably performed with an interval of at least 2 days. If the subject is poultry, in one embodiment, one of the administrations is in ovo administration and one of the administrations is administration after hatch, preferably within one week after hatch, more preferably within 3 days after hatch. If the subject is a mammal, such as a human, a bovine, including dairy and beef cattle, other ruminant including a sheep, a pig, a dog, a cat or a horse, the second administration can be performed e.g. between 2 and 20 days after the first administration.

The composition comprising the CATH2 or derivative used in accordance with the invention may further comprise a pharmaceutically acceptable carrier, preferably a veterinary acceptable carrier. Such acceptable carrier may include solvents, dispersion media, coatings, adjuvants, stabilizing agents, diluents, preservatives, antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

Adjuvants suitable for use in the present method include but are not limited to: mineral gels, e.g., aluminum hydroxide; surface active substances such as lysolecithin; glycosides, e.g., saponin derivatives such as Quil A or GPI-0100 (U.S. Pat. No. 5,977,081); cationic surfactants such as DDA, pluronic polyols; polyanions; non-ionic block polymers, e.g., Pluronic F-127 (B.A.S.F., USA); peptides; mineral oils, e.g. Montanide ISA-50 (Seppic, Paris, France), carbopol, Amphigen (Hydronics, Omaha, Nebr. USA), Alhydrogel (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol, rmLT, cytokines and combinations thereof. The immunogenic component may also be incorporated into liposomes, or conjugated to polysaccharides and/or other polymers for use in a vaccine formulation. Additional substances that can be included in a product for use in the present methods include, but are not limited to one or more preservatives such as disodium or tetrasodium salt of ethylenediaminetetracetic acid (EDTA), merthiolate, and the like. Immunostimulants which enhance the immune system's response to antigens may also be included in a product. Examples of suitable immunostimulants include cytokines such as IL-12 or IL-2, or stimulatory molecules such as muramyl dipeptide, aminoquinolones, lipopolysaccharide, and the like.

Because the present inventors have found that the CATH2 derivatives as described herein induce trained immunity, it is particularly advantageous to combine the derivative with an adjuvant for innate immune cells. Hence, in one preferred embodiment, the adjuvant is an adjuvant for innate immune cells, i.e. basophils, dendritic cells, eosinophils, Langerhans cells, mast cells, monocytes and macrophages, neutrophils and/or NK cells, as described herein above: TLR) ligands, β-glucan, muramyl dipeptide (MDP) or peptide comprising MDP, Bacille Calmette-Guerin (BCG) and CpG containing oligodeoxynucleotide.

In one embodiment, the composition comprising the CATH2 or derivative used in accordance with the invention comprises a buffered solution, such as a phosphate buffered saline (PBS) solution, or cholesterol. In one embodiment, composition comprising the CATH2 or derivative used in accordance with the invention comprises a buffered solution, such as a phosphate buffered saline (PBS) solution, and cholesterol. For instance, the cholesterol is first solubilized in ethanol, mixed with PBS and then mixed with the CATH2 or derivative, preferably in dissolved form, resulting in a particulate composition. In one embodiment, prior to administration the dissolved CATH2 or derivative is mixed with a cholesterol solution to form fine particulates and subsequently administered.

A pharmaceutical composition for use in accordance with any method or use of the present invention comprises an effective amount of CATH2 or derivatives as defined herein. As used herein the term “effective amount” refers to an amount of CATH2 or derivative being administered that is sufficient to inducing or promoting innate immune memory in a subject in need thereof as defined herein.

In one embodiment, the composition comprises a therapeutically effective amount of the CATH2 or derivative thereof. The term “therapeutically effective amount,” as used herein, refers to an amount of CATH2 or derivative being administered sufficient to relieve one or more of the symptoms of the disease or condition being treated to some extent, in particular of a S. suis infection. This can be a reduction or alleviation of symptoms, reduction or alleviation of causes of the disease or condition or any other desired therapeutic effect. In particular, the therapeutically effective amount refers to an amount of CATH2 or derivative being administered sufficient to inhibit S. suis, wherein inhibition of S, suis is as defined herein above.

In one embodiment, the composition comprises a prophylactically effective amount of the CATH2 or derivative thereof. As used herein, the term “prophylactically effective amount” refers to an amount of CATH2 or derivative being administered sufficient to preclude or delay the onset of a disease or condition and/or the appearance of clinical symptoms of the disease or condition in a subject that does not yet experience clinical symptoms of the disease, in particular of a S. suis infection, In particular, the prophylactically effective amount refers to an amount of CATH2 or derivative being administered sufficient to prevent S. suis infection.

The pharmaceutical composition may also comprise CATH2 and one or more derivatives as defined herein, or it may comprise two or more CATH2 derivatives as defined herein, such as a combination of DCATH2 and D(1-21).The “effective amount”, “therapeutically effective amount” and “prophylactically effective amount” in that case refer to the combined amount of the two or more of CATH2 and/or one or more derivatives thereof.

Effective amounts or dosages of the CATH2 or derivative required for use in a method or use of the invention, can easily be determined by the skilled person, for instance by using animal models. For purposes of the present invention, an effective dose will be from about 0.01 μg/kg to 50 mg/kg, preferably 0.5 μg/kg to about 10 mg/kg of the CATH2 or derivative thereof in the subject to which it is administered. For in ovo applications the same doses may be used, but recalculated with relation to the weight of the embryo.

The pharmaceutical composition used in accordance with any method or use of the present invention may also comprise one or more pharmaceutically acceptable excipients. By “pharmaceutically acceptable” it is meant that the carrier, diluent or excipient must be compatible with the other ingredients of the formulation and not deleterious, e.g. toxic, to the recipient thereof. In general, any pharmaceutically suitable additive which does not interfere with the function of the active compounds can be used. Suitable examples of excipients are a carrier or a diluent. The pharmaceutical compositions may be in the form of a capsule, tablet, lozenge, dragee, pill, droplet, suppository, powder, spray, vaccine, ointment, paste, cream, inhalant, patch, aerosol, and the like. As pharmaceutically acceptable carrier, any solvent, diluent or other liquid vehicle, dispersion or suspension aid, surface active agent, isotonic agent, thickening or emulsifying agent, preservative, encapsulating agent, solid binder or lubricant can be used which is most suited for a particular dosage form and which is compatible with the CATH2 or derivative.

Salts of the CATH2 or derivative may also be used. In one preferred embodiment, pharmaceutically acceptable salts are used. Salts of peptides can be prepared by known methods, which typically involve the mixing of the peptide with either a pharmaceutically acceptable acid to form an acid addition salt, or with a pharmaceutically acceptable base to form a base addition salt. Whether an acid or a base is pharmaceutically acceptable can be easily decided by a person skilled in the art after taking the specific intended use of the peptide into consideration. Pharmaceutically acceptable acids include organic and inorganic acids such as formic acid, acetic acid, propionic acid, lactic acid, glycolic acid, oxalic acid, pyruvic acid, succinic acid, maleic acid, malonic acid, cinnamic acid, sulfuric acid, hydrochloric acid, hydrobromic acid, nitric acid, perchloric acid, phosphoric acid, and thiocyanic acid, which form ammonium salts with free amino groups of peptides and functional equivalents. Pharmaceutically acceptable bases, which form carboxylate salts with free carboxylic groups of peptides and functional equivalents, include ethylamine, methylamine, dimethylamine, triethylamine, isopropylamine, diisopropylamine, and other mono-, di- and trialkylamines, as well as arylamines.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of CATH2 or derivative in either an oil or in aqueous propylene glycol may be employed. The aqueous solutions can be suitably buffered and the liquid diluent can be rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques known to those skilled in the art.

Additionally, it is also possible to administer the CATH2 or derivative topically and this may be done by way of creams, jellies, gels, pastes, patches, ointments and the like, in accordance with standard pharmaceutical practice.

Once formulated, the pharmaceutical compositions can be administered directly to the subject in a method or use in accordance with the invention. Direct delivery of the compositions will generally be accomplished by forms of administration, including orally, parenterally, subcutaneously, sublingually, intraperitoneally, intravenously or intramuscularly, pulmonary. Such administration may be carried out in single or multiple doses.

It may also be advantageous to administer the CATH2 or derivative in a transmucosal dosage form. This route of administration is non-invasive and thus less cumbersome for the subject that is being treated and at the same time it may lead to an improved bioavailability compared to oral administration, especially if the compound is not stable in the fluids of the digestive system, or if it is too large to be absorbed from the gut effectively. Transmucosal administration is possible, for instance, via nasal, buccal, sublingual, gingival, or vaginal dosage forms. These dosage forms can be prepared by known techniques; they can be formulated to represent nasal drops or sprays, inserts, films, patches, gels, ointments, or tablets. Preferably, the excipients used for a transmucosal dosage form include one or more substances providing for mucoadhesion, thus prolonging the contact time of the dosage form with the site of absorption and thereby potentially increasing the extent of absorption.

In one embodiment, the CATH2 or derivative is administered via the pulmonary route, using a metered dose inhaler, a nebulizer, an aerosol spray, or a dry powder inhaler. Appropriate formulations can be prepared by known methods and techniques. Transdermal, rectal, or ocular administration may also be feasible in some cases.

Pharmaceutical compositions administered in accordance with the invention may contain other active agents, such as conventional antibiotics (like e.g. vancomycin, streptomycin, tetracyclin, penicillin) or other antimicrobial compounds, such as anti-fungals, e.g. itraconazole or myconazole. Also compounds that alleviate other infection symptoms, such as fever (e.g. salicylic acid) or skin rash may be added.

The CATH2 or derivative used in accordance with the invention can be produced synthetically or, where applicable, recombinantly by conventional methods. Suitable methods are well known in the art and for instance described in van Dijk, A., et al., Identification of chicken cathelicidin-2 core elements involved in antibacterial and immunomodulatory activities (Mol Immunol, 2009. 46(13): p. 2465-73, reference [6]) and van Dijk, A., et al., Immunomodulatory and Anti-Inflammatory Activities of Chicken Cathelicidin-2 Derived Peptides (PLoS One, 2016. 11(2): p. e0147919, reference [7]), which are incorporated herein by reference. Preferably, the CATH2 or derivatives of the invention are prepared conventionally by known chemical synthesis techniques, such as, for instance, are disclosed by Merrifield (J. Am. Chem. Soc. (1963) 85:2149-2154). They may be isolated from the reaction mixture by chromatographic methods, such as reverse-phase HPLC.

Alternatively, CATH2 or derivative used in accordance with the invention may be produced by recombinant DNA techniques by cloning and expressing within a host micro-organism or cell a DNA fragment carrying a nucleic acid sequence encoding one of the above-described peptides. Nucleic acid coding sequences can be prepared synthetically, or may be derived from existing nucleic acid sequences (e.g. the sequence coding for wild-type CATH2) by site-directed mutagenesis. These nucleic acid sequences may then be cloned in a suitable expression vector and transformed or transfected into a suitable host cell, such as E. coli, Bacillus, Lactobacillus, Streptomyces, mammalian cells (such as CHO, HEK or COS-1 cells), yeasts (e.g. Saccharomyces, Schizophyllum), insect cells or viral expression systems, such as baculovirus systems, or plant cells. A person skilled in the art will have knowledge of the techniques of constructing the nucleic acid sequences and providing means to enable their expression. Subsequently, the CATH2 or derivative can be isolated from the culture of the host cells. This can be achieved by common protein purification and isolation techniques, which are available in the art. Such techniques may e.g. involve immunoadsorption or chromatography. It is also possible to provide the peptides with a tag (such as a histidine tag) during synthesis, which allows for a rapid binding and purification, after which the tag is enzymatically removed to obtain the active peptide. Alternatively, the CATH2 or derivative can be produced in cell-free systems, such as the Expressway™ cell-free system of Invitrogen.

Some more comprehensive summaries of methods which can be applied in the preparation of peptides, i.e. including CATH2 or derivative thereof, are described in: W. F. Anderson, Nature 392 Supp., 30 Apr. 1998, p. 25-30; Pharmaceutical Biotechnology, Ed. D. J. A. Crommelin and R. D. Sindelar, Harwood Academic Publishers, 1997, p. 53-70, 167-180, 123-152, 8-20; Protein Synthesis: Methods and Protocols, Ed. R. Martin, Humana Press, 1998, p. 1-442; Solid-Phase Peptide Synthesis, Ed. G. B. Fields, Academic Press, 1997, p. 1-780; Amino Acid and Peptide Synthesis, Oxford University Press, 1997, p. 1-89.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: _{D}-CATH2 and its derivatives efficiently kills several S. suis type2 strains in both THB and RPMI+FCS. Antibacterial activity of _{D}-CATH2 and its derivatives against 10⁶ CFU ml⁻¹ S. suis type 2 strains (P1/7, D282, 5735, and OV625) was tested using a colony count assay in both THB medium (A) and RPMI+FCS (B). 2 log CFU mL⁻¹ was set as detection limit for the experiment. Data is plotted as average +/−SEM (N=3-4).

FIG. 2: Peptide titration on BMDM and BMDCs for cytotoxicity and expression of cell markers. Mouse BMDM (A, C, E, G) and BMDCs (B, D, F, H) were cultured for 6 days with GM-CSF and M-CSF respectively. Different concentrations were added at day 6 (A, B, E, F) or the cells were primed with different concentrations peptides at day 1-2 (C, D, G, H). At day 7, cell viability was tested using WST-1 reagent, with no-peptide-control set to 100% viability (A-D) and cells were analyzed by flowcytometry for cell marker expression (E-H). Results are depicted as mean +/−S.E.M. (n=3).

FIG. 3: _{D}-CATH2 and its derivatives inhibit LTA-SA- or S. suis-induced activation. Mouse BMDM cells were cultured for 6 days before they were activated with different S. suis type2 strains at an MOI of 0.2. Bacteria were mixed for 5 minutes with 1.25 μM _{D}-CATH2 or its derivatives before stimulation. After 24 hours of stimulation, cells were analyzed by flowcytometry (A) and cytokine expression was measured (B). 5*10⁵ splenocytes, freshly isolated from WT mice using a digestion buffer followed by filtering through a 40 μM cell filter, were activated with different S. suis type2 strains premixed with 5 μM _{D}-CATH2 or its derivatives at an MOI of 0.2. After 24 hours of stimulation, secreted cytokines were measured using ELISA (C). Data is plotted as average +/−SEM (N=3-6).

FIG. 4: _{D}-CATH2 and its derivatives bind to LTA.

Thermodynamic binding capacity of 200 μM _{D}-CATH2 (A), _{D}C(1-21) (B), and _{D}C(4-21) (C) to 200 μM LTA-SA was measured using isothermal titration calorimetry (ITC). Every 300 seconds, 1.99 μl peptide solution was titrated into 164 μl LTA solution. The corrected heat rate (μJ sec⁻¹) is plotted (top panel) and normalized integrated heat was plotted against the molar ratio between LTA and the peptide (lower panel). Experiments (N=3) were averaged before plotting and fitting an independent model. The corrected heat rate of _{D}-CATH2, _{D}C(1-21), and _{D}C(4-21) is depicted for comparison (D).

FIG. 5: _{D}-CATH2 and its derivatives increase the BMDM efficiency.

Mouse BMDM cells were cultured for 6 days. At day 1, 1.25 μM _{D}-CATH2 or its derivatives were added for 24 hours. At day 6 the cells were activated with different S. suis type2 strains at an MOI of 0.2. Bacteria were mixed for 5 minutes with 1.25 μM _{D}-CATH2 or its derivatives before stimulation. After 24 hours of stimulation, cells were analyzed by flowcytometry (A) and cytokine expression was measured (B). Data is plotted as average +/−SEM (N=3-6).

FIG. 6: Dendritic cell primed by peptides have increased macrophage markers. Mouse BMDCs were cultured for 6 days with M-CSF and either primed with 1.25 μM peptides at day 1-2 (A) or 1.25 μM peptide was added at day 6 during stimulation (B). Cells were analyzed by flowcytometry for cell marker expression. Results are depicted as mean +/−S.E.M. (n=3).

FIG. 7: Prophylactic _{D}C(1-21) s.c. injection reduces the clinical symptoms of S. suis P1/7 in mice.

A schematic overview of the in vivo experiment set up. At day 1, all mice were subcutaneously injected with _{D}C(1-21) or a control in the neck region. Either after 24 hours (24 h _{D}C(1-21) or 7 days (7 d _{D}C(1-21) the mice were intra-peritoneally injected with 10⁷ CFU S. suis P1/7 or only THB. 24 hours after infection, a few drops of blood were collected and 7 days post infection, the mice were sacrificed for analysis. The black arrows indicate the moment of animal welfare evaluation by weighing and score for clinical symptoms (A). The relative weight difference with the weight at the moment of infection is set to 100% is depicted for 24 h _{D}C(1-21) (B) and 7 d _{D}C(1-21) (E). The cumulative clinical score of 8 different parameters is depicted for 24 h _{D}C(1-21) (C) and 7 d _{D}C(1-21) (F). Survival curves are depicted and bacterial counts in different organs of mice reaching HEP is depicted for 24 h _{D}C(1-21) (D) and 7 d _{D}C(1-21) (G). The number of organs per mouse in which S. suis bacteria were found (H) and the average CFU per organ per mouse (I). The bacterial burden of mice died before the end of the study with circles depicting mice infected 24 hours post peptide injection and squares 7 days post peptide injection (J). Results are depicted as mean +/−S.E.M. (CNTR n=4, CNTR+S.suis n=12, _{D}C(1-21) n=4, and _{D}C(1-21)+S. suis n=12).

FIG. 8: Bacterial counts in the blood and different organs of S. suis infected mice. 24 hours post infection, blood was drawn via cheek puncture (A) and via heart puncture after 7 days (B) and plated on TSA/5% sheep blood plates for bacterial counts (A,B). The peritoneum was flushed at day 7 and cells present in the peritoneal lavage (PTL) were counted (C). The spleens were weighed (D). The single cell suspension of the different organs was plated on TSA/5% sheep blood plates for bacterial counts. CFU per mg organs was calculated (E).

### EXAMPLES

### Materials and Methods

### Peptides, Bacterial Strains and Experimental Animals

The 26 amino acid full _{D}-antiomer of chicken CATH2 (RFGRFLRKIRRFRPKVTITIQGSARF-NH₂) (_{D}-CATH2) with a net positive charge of 9 (9+) and two derivatives (_{D}C(1-21), 8+ and _{D}(4-21), 7+) were used in this study. The peptides were synthesized by Fmoc-chemistry at China Peptides (CPC scientific, Sunnyvale, CA, USA) and purified by reverse phase high-performance liquid chromatography to a purity of >95%. Lyophilized peptides were dissolved in endotoxin free water.

S. suis Serotype 2 strain P1/7, D282, 5735, and OV625 were used in this study. All strains have been previously characterized.[17] Bacterial strains were grown overnight from glycerol stocks in Todd-Hewitt broth (THB) (Oxoid Ltd., London, UK) before use.
Seven- to ten-week-old Crl:CD-1 mice (both male and female) were purchased from Charles River. All mice were kept under specific pathogen-free conditions with free access to food and water under the guidelines for animal experimentation as approved by the Dutch central authority for scientific procedures on animals (CCD).

### Antibacterial Activity

S. suis Serotype 2 strains P1/7, D282, S735, and OV625 were grown into mid-logarithmic phase for 3-4 hours at 37° C. in THB, after which bacteria were centrifuged at 1200×g for 10 minutes at 4° C. and resuspended in fresh THB. Concentration was determined by measuring the OD value at 620 nm with an OD of 0.1 is 1×10⁸ colony forming units (CFU) mL⁻¹. 10⁶ CFU mL⁻¹ S suis was mixed with different concentrations of _{D}-CATH and derivatives (0.63−40 μM) and left for 3 hours at 37° C. Ten-fold dilutions were prepared and spread in Tryptan Soy agar (TSA) plates containing 5% (vol/vol) defibrinated sheep blood (Oxoid) and colonies were allowed to grow for 48 hours. Minimal Bactericidal Concentration (MBC) was defined as ≤100 CFU mL⁻¹ (2 logCFU mL⁻¹), the detection limit of the assay.

### Cell Culture and Flow Cytometry

Bone marrow cells, isolated from the femur and tibia of both hindlegs, were stored in FCS/10% DMSO in liquid nitrogen. Cells were grown at a concentration of 5×10⁵ cells mL⁻¹ in RPMI-1640 without phenol red (Thermo Fisher Scientific, MA, USA) supplemented with 10% fetal calf serum (FCS) (Corning, NY, USA) and 1% Penicillin/streptomycin (Thermo Fisher Scientific). Bone marrow derived macrophages (BMDM) and bone marrow derived dendritic cells (BMDC) were culture by adding 20 ng mL⁻¹ murine recombinant M-CSF or GM-CSF (Peprotech, NJ, USA) respectively. If indicated, cells were trained by adding 1.25 μM peptide at day 1, which was replace by fresh medium at day 2. The medium of all cells was replaced by fresh medium without antibiotics at day 3. At day 6 cells were stimulated with 1 μg mL⁻¹ lipoteichoic acid from S. aureus (LTA-SA) (Invivogen) or with the different S suis strains with a multiplicity of infection (MOI) of 0.2. Medium containing S. suis was removed after 2 hours and replaced by medium containing 200 μg/ml gentamycin (Sigma-Aldrich, MO, USA) and left for an additional 22 hours. After 24 hours, medium was collected and stored at −20° C. for cytokine measurements. Cells were incubated for 5 min with PBS/0.5 mM EDTA after which they were resuspended by vigorous pipetting and used for flow cytometry. Cells were resuspended in flow cytometry buffer (PBS/0.5% BSA (Sigma Aldrich)) and kept on ice during the whole procedure. Cells were stained with antibodies (table 1) for 20 minutes, washed and measured using the BD FACSCanto-II (BD bioscience) and analyzed with FlowJo software (Ashland, OR, USA).

### Splenocytes Activation

Mice were killed using CO₂ suffocation after which the spleen were harvested. Spleen were digested with digestion buffer (1.5 WU/ml liberase TL grade (Roche, Basel, Switzerland), 100 Units/ml recombinant DNAse I (Roche) for 30 minutes at 37° C. and meshed through a 40 μm filter (BD bioscience) to prepare single cell solution using PBS/0.5 mM EDTA wash buffer. The red blood cells were lysed using an isotonic ammonium chloride buffer (155 mM NH₄Cl, 10 mM KHCO₃, 0.1 mM EDTA) for 5-10 minutes on ice, washed 1× with PBS, after which the cells were counted and resuspended in in high glucose DMEM (Thermo Fisher scientific, MA, USA) supplemented with 10% FCS (Corning, VA, USA). 5×10⁵ splenocytes were added per well in a U-bottom 96-wells plate. Total splenocytes were stimulated with 1 μg LTA-SA or the different S. suis strains at an MOI of 0.2. After 2 hours, the supernatant was collected (by centrifugation 1800 RPM, 2 min) and the cells were resuspended in 100 μl fresh medium supplemented with 200 μg/ml gentamycin and left for an additional 22 hours. After 24 hours, medium was collected and stored at −20° C. for cytokine measurements.

### Cell Viability and Activity

WST-1 reagent (roche) was used for cell viability of BMDCs and BMDM as well as for cell activity of activated splenocytes. In both cases, 100 μl fresh medium containing 10% WST-1 was added and incubated at 37° C. After 30-60 minutes, colorimetric changes were measured at 450 nm using a FLUOstar Omega microplate reader (BMG Labtech GmbH, Ortenberg, Germany). The metabolic activity is depicted as a percentage with the untreated BMDCs/BMDMs or unstimulated splenocytes set to 100%.

### ELISA

TNFα, IFNγ, IL-1β, and IL-6 were measured in the supernatant (diluted in PBS/5% BSA if needed) using a Duoset ELISA kit (R&D systems, MN, USA). ELISAs were performed according manufacturer's instructions. Colorimetric changes were measured at 450 nm using a FLUOstar Omega microplate reader (BMG Labtech GmbH).

### Isothermal Calorimetry (ITC)

The interaction between the _{D}-CATH2 peptides and LTA-SA was tested using isothermal titration calorimetry (ITC). All ITC experiments were performed on a Low Volume NanoITC (TA instruments-Waters LLC, New Castle, USA). 800 μM of LTA-SA or peptide solution was prepared in MilliQ after which a 4-fold dilution in dPBS (Gibco) was made. The chamber was filled with 164 μl LTA-SA and the peptide was loaded in the syringe. Every 300 seconds, 1.99 μL peptide was titrated into the chamber at 37° C. Data was analyzed using the Nano Analyze software (TA instruments-Waters LLC). The data of three experiments was averaged and an independent model was used to determine the peptide-LTA interaction.

### In Vivo Infection Experiment

Upon arrival, mice were allowed to acclimate for at least 7 days before the start of the experiment. The experiment was performed as depicted in FIG. 7A. The experiment was repeated twice to obtain in total 4 mice in the control groups and 12 mice in the infection groups. At day 1, mice were s.c. injected in the neck region with 1 mg kg⁻¹ _{D}C(1-21) in PBS/Cholesterol or with PBS/cholesterol alone. The peptide and control groups were blinded to avoid any influence by the researchers. After 24 hours (group 1) or after 7 days (group 2), mice were i.p. infected with 10⁷ CFU S. suis P1/7 in THB or with THB alone as control. 24 hours after infection, a few drops of blood were collected via cheek puncture for bacterial count. During the infection phase of the experiment, mice were checked every 12 hours in the acute phase of disease (first 48 hours) and thereafter daily until the end of the end of the study. A cumulative clinical score was given to the mice as measure of disease using several parameters as depicted in table 4, according to Seitz et al.[18] When a mouse obtained a clinical score of 2 on 3 of the 8 points 2 days in a row or in case of severe weight loss (>20%), the mouse was euthanized for animal welfare reasons (humanized end point (HEP)) and the organs were collected for bacterial counts as described hereafter. 7 days post infection all mice were sacrificed for further analysis. Mice were anesthetized using Isoflurane and 1 mL blood was drawn via heart puncture, followed by cervical dislocation. The peritoneum was flushed with 5 mL PBS/0.5 mM EDTA and diluted in 10 mL ice cold PBS/0.5% FCS. The organs (spleen, lungs, liver, lymph nodes (axillary, inguinal, and mesenteric), brain, kidney and bone marrow) were collected and stored in ice cold PBS. All organs, except the bone marrow and lymph nodes were weight using a sartorius microbalance. The peritoneal lavage (PTL) samples were counted using the Countess II Automated Cell Counter (Thermofisher). The lungs, liver, brain and kidney were meshed through a 40 μm filter (BD bioscience) with 5 mL PBS to obtain single cell suspensions. Spleen and lymph nodes were digested with digestion buffer (1.5 WU/ml liberase TL grade (Roche, Basel, Switzerland), 100 Units/ml recombinant DNAse I (Roche) for 30 minutes at 37° C. and meshed through a 40 μm filter using 5 mL PBS/0.5 mM EDTA. The red blood cells of the blood and spleen were lysed using an isotonic ammonium chloride buffer (155 mM NH₄Cl, 10 mM KHCO₃, 0.1 mM EDTA) for 5-10 minutes on ice, washed 1× with PBS and were resuspended in FACS buffer (PBS/0.5% BSA). Bone marrow samples were flushed out the femur and tibia of both legs with 5 mL PBS and filtered through a 40 μm filter. A sample of the blood, bone marrow, spleen, peritoneal lavage and lymph nodes was taken and stained for 30 minutes with different antibody panels (table 1) and measured using the BD FACSCanto-II and analyzed with FlowJo software. Of the lungs, liver, brain, kidney, spleen and peritoneal lavage samples, a 10-fold serial dilution was prepared and the samples were plated on TSA plates containing 5% (vol/vol) defibrinated sheep blood. The colonies were allowed to grow for 48 hours. The number of colonies were counted, with ≤100 CFU mL-1 (2 logCFU mL-1) as detection limit of the assay, and calculated as CFU/mg organ.

TABLE 1
Antibodies used

| Antigen | Clone | Label | Manufacture |
|---|---|---|---|
| MHC-II | M5/114.15.2 | FITC | eBioscience |
| CD11c | HL3 | PE | BD Bioscience |
| Sirp-α | P84 | PerCP- | eBioscience |
| | | eFluor710 | |
| CD19 | 1D3 | PE-Cy7 | BD Bioscience |
| CD8α | 53-6.7 | APC | BD Bioscience |
| CD11b | M1/70 | APC-Cy7 | BD Bioscience |
| CD24 | M1/69 | eFluor450 | eBioscience |
| CD86 | GL-1 | PerCP | BoiLegend |
| F4/80 | BM8 | APC | eBioscience |
| Ly6C | HK1.4 | eFluor450 | eBioscience |
| CD4 | RM4-5 | AF488 | BD |
| | | | Pharmingen |
| CD62L | MEL-14 | PE | eBioscience |
| CD335 | 29A1.4 | PerCP-Cy5.5 | BD Bioscience |
| CD44 | IM7 | PE-Cy7 | BD Bioscience |
| CD3e | 145-2C11 | APC-Cy7 | BD |
| | | | Pharmingen |
| CD25 | eBio3C7 | eFluor450 | eBioscience |

Antibodies used for flow cytometry. All antibodies were diluted 1000x in flow cytometry buffer prior to use (CD19 and CD335 were used in a 500x dilution).

### Statistics

Samples were compared to no-peptide-controls using two-way ANOVA with the Dunnett post-hoc test. Samples were paired for cell culture samples. *=p≤0.05; **=p≤0.01; ***=p≤0.001;****=p≤0.0001.

### Results

### _{D}-CATH2 and its Derivatives Efficiently Kills Several S. Suis Type2 Sub-Strains in Both THB and RPMI+FCS

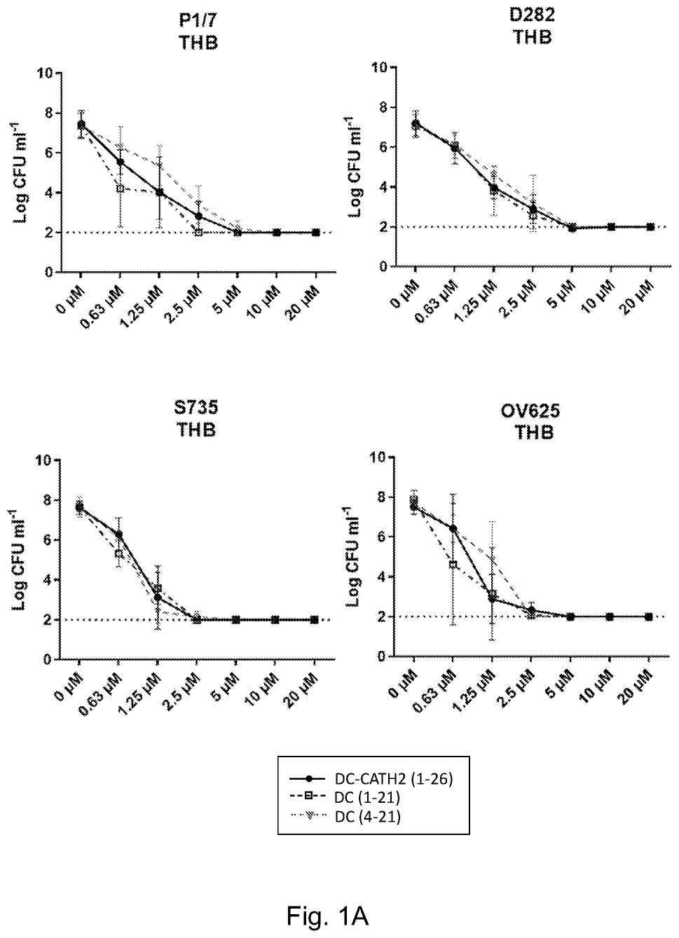

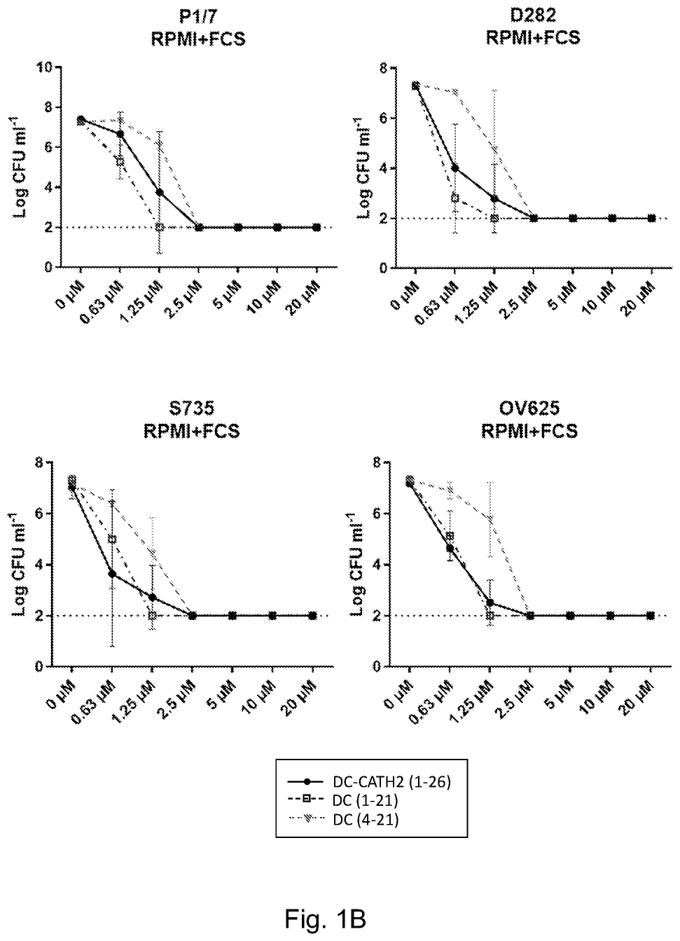

Antimicrobial activity of d-CATH2 and its derived peptides was assessed against 4 different S. suis serotype 2 strains. The mean bactericidal concentration (MBC) of the three peptides is 2.5-5 μM for the four sub-strains in bacterial growth medium THB (FIG. 1A and Table 2). However, most of the assays will be performed in cell culture medium RPMI+10% FCS and it has been shown that medium can influences the activity of cathelicidins,[19,20] the MBC of _{D}-CATH2 and its derived peptides was also tested in RPMI+10% FCS medium. The activity of _{D}-CATH2 and _{D}C(1-21) slightly increased to 0.6-2.5 μM, whereas the shortest peptide, _{D}C(4-21), remained with a MBC of 2.5 μM (FIG. 1B and Table 2), indicating that either charge or number of amino acids is important for the antibacterial function of _{D}-CATH2.

TABLE 2
MBC values of D-CATH2 killing S. suis strains

| | THB | | | RPMI + 10% FCS | | |
|---|---|---|---|---|---|---|
| | D- | DC | DC | D- | DC | DC |
| | CATH2 | (1-21) | (4-21) | CATH2 | (1-21) | (4-21) |
| P1/7 | 2.5-5 | 2.5 | 5-10 | 1.25-2.5 | 1.25 | 2.5 |
| S735 | 1.25-2.5 | 2.5 | 2.5 | 0.6-2.5 | 1.25 | 2.5 |
| D282 | 2.5-5 | 2.5-5 | 2.5-5 | 0.6-2.5 | 0.6-1.25 | 1.25-2.5 |
| OV625 | 1.25-5 | 0.6-5 | 1.25-2.5 | 1.25-2.5 | 1.25 | 2.5 |

MBC values for the different peptides depending on the bacterial strain and the medium.

### _{D}-CATH2 and its Derivatives Inhibit LTA-SA- or S. Suis-Induced Activation by Binding to LTA

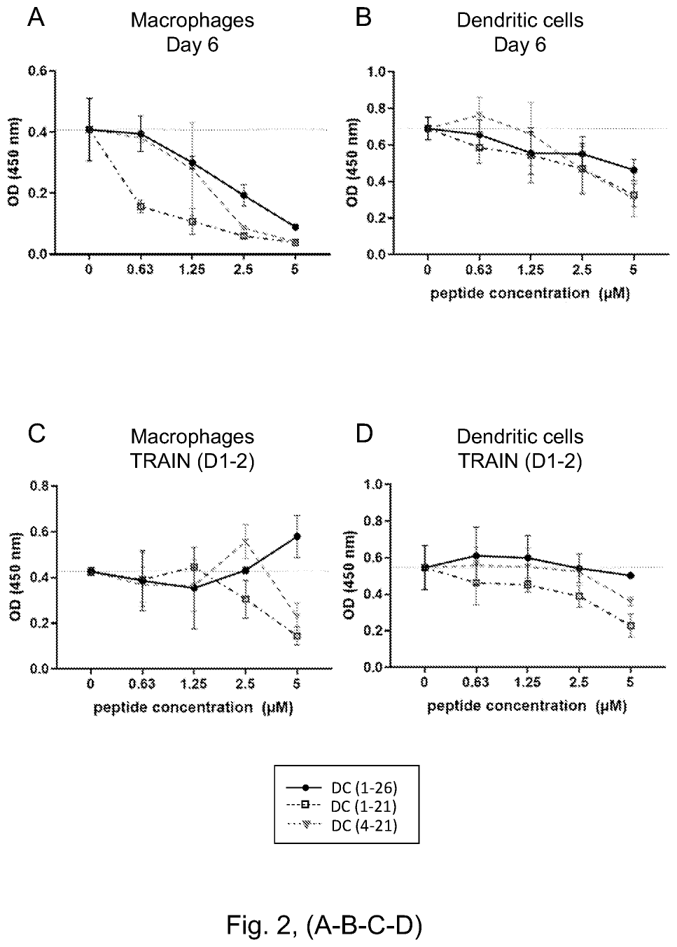

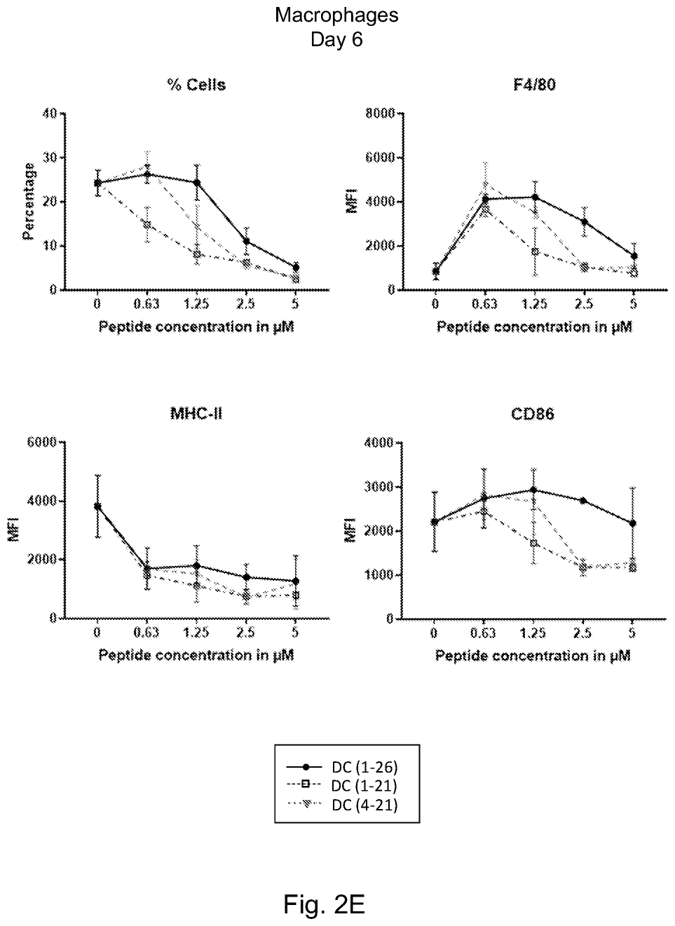

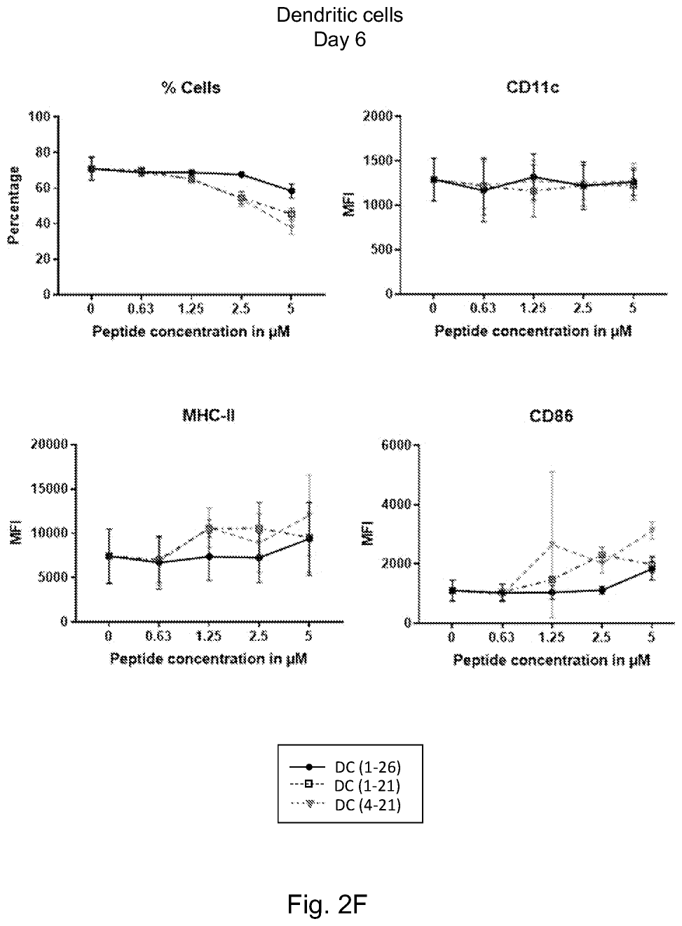

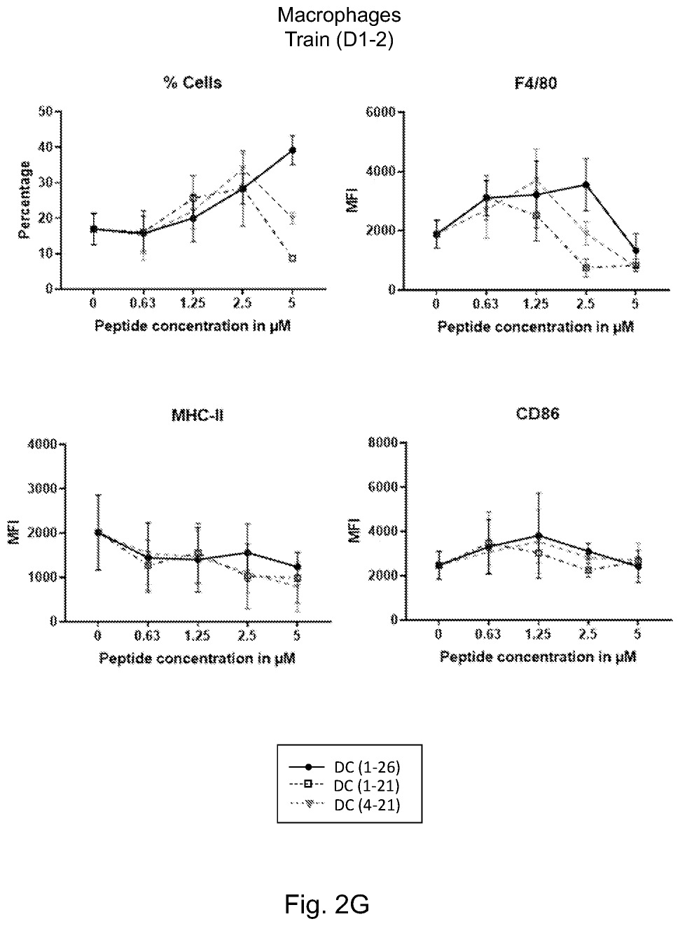

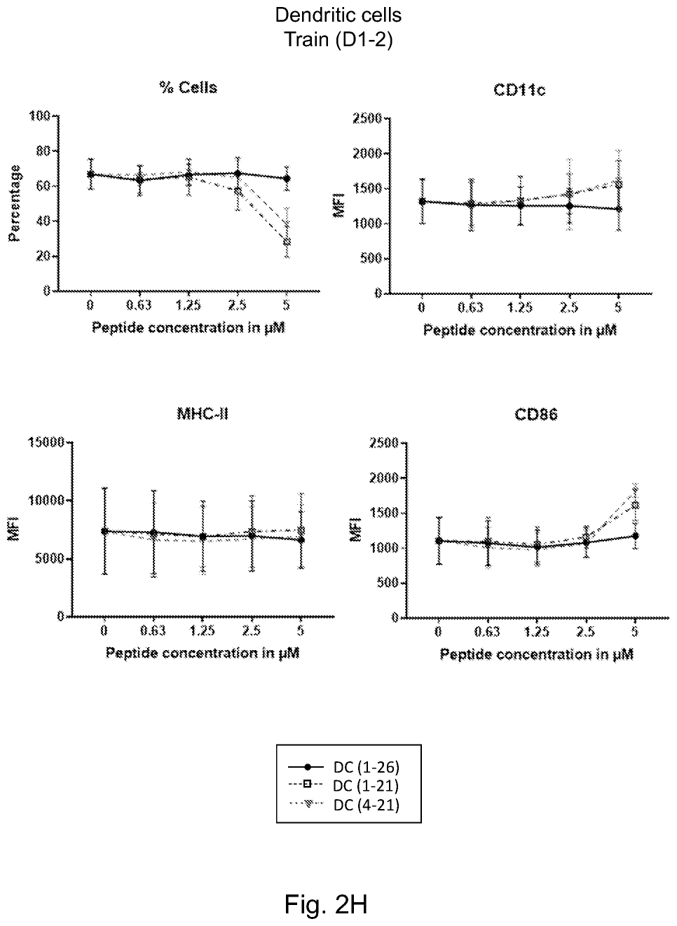

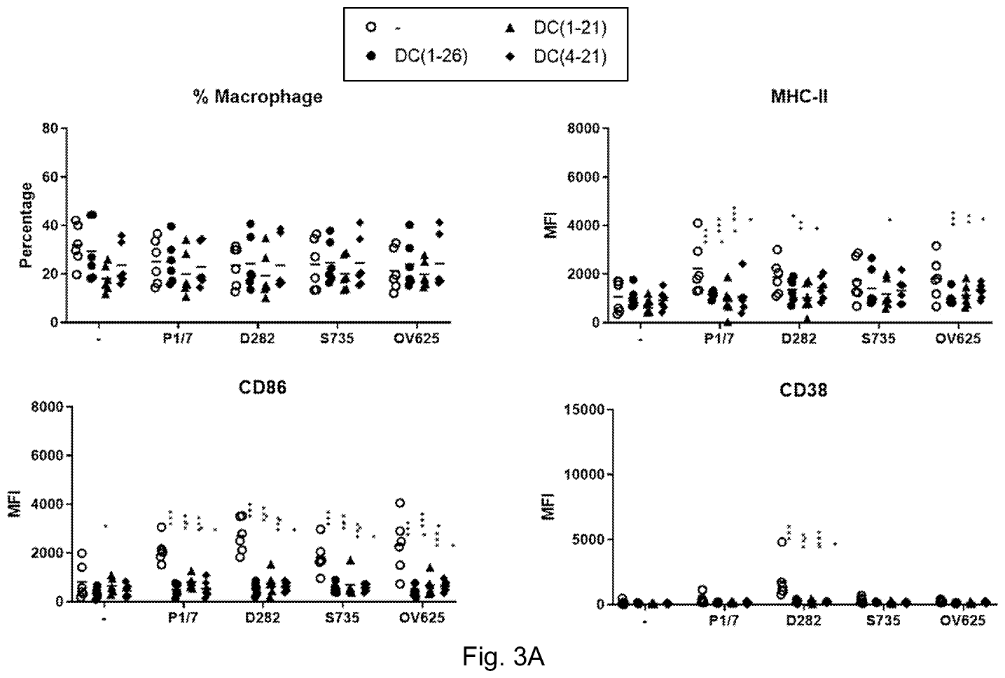

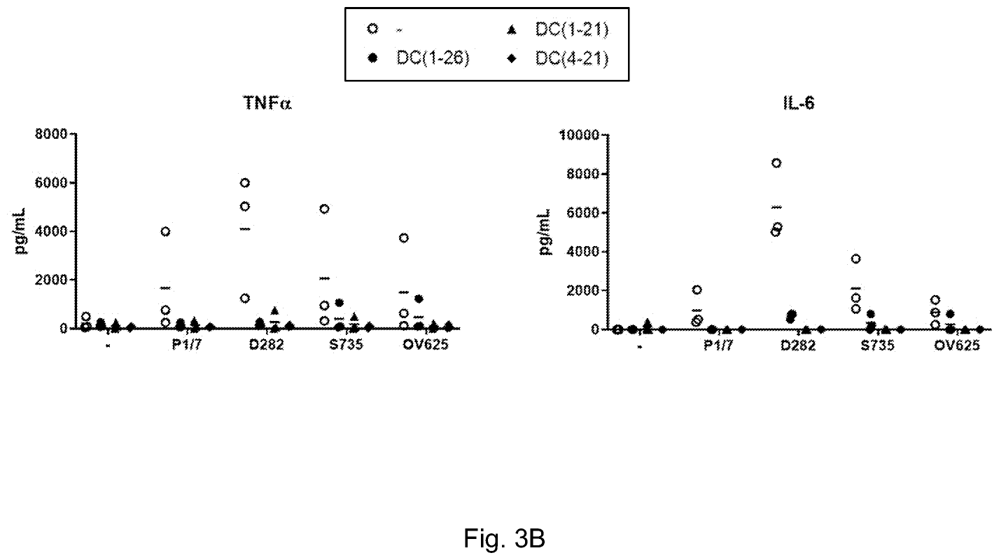

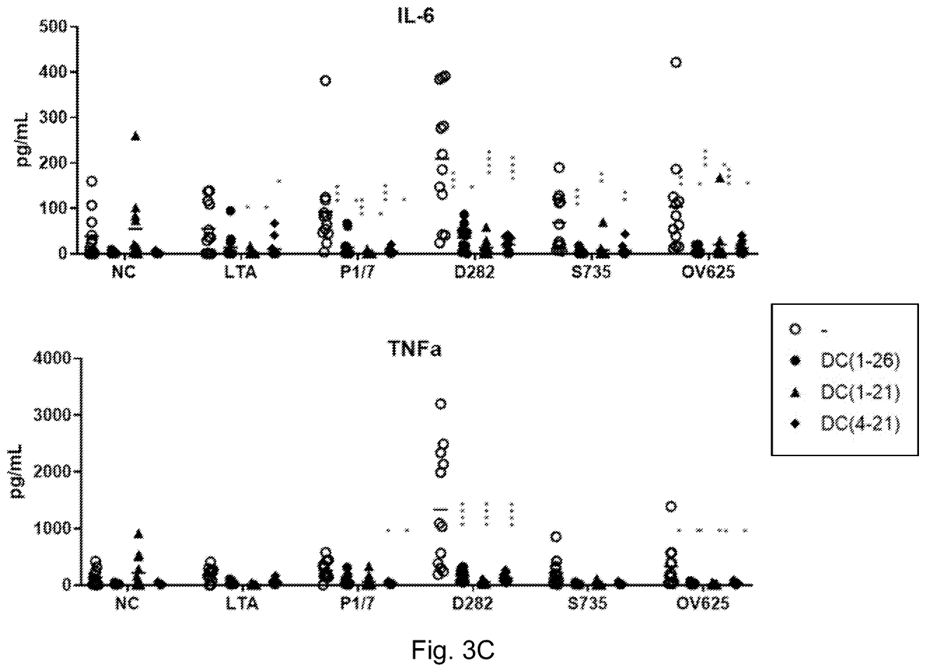

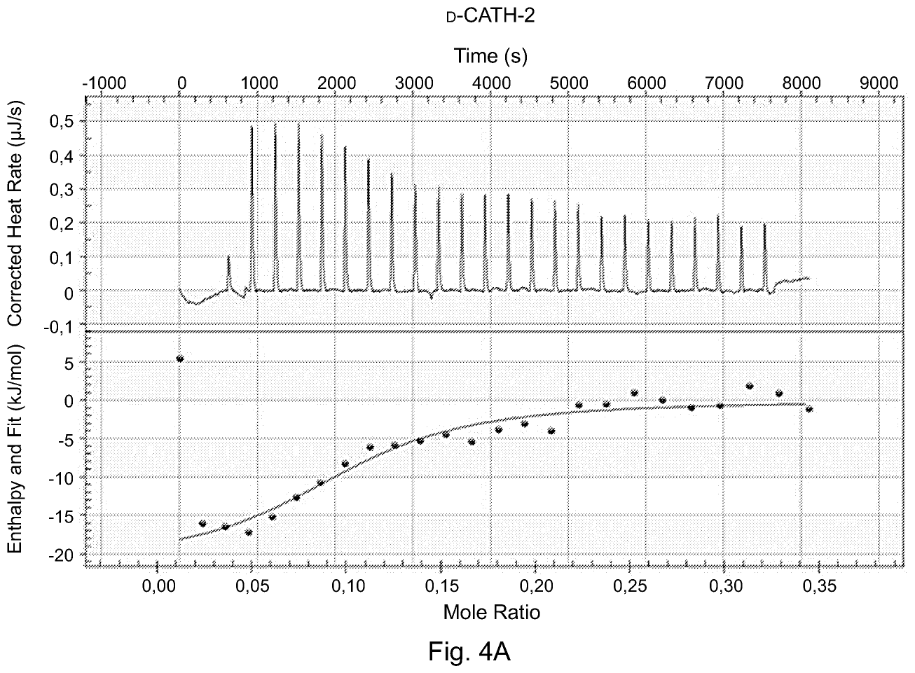

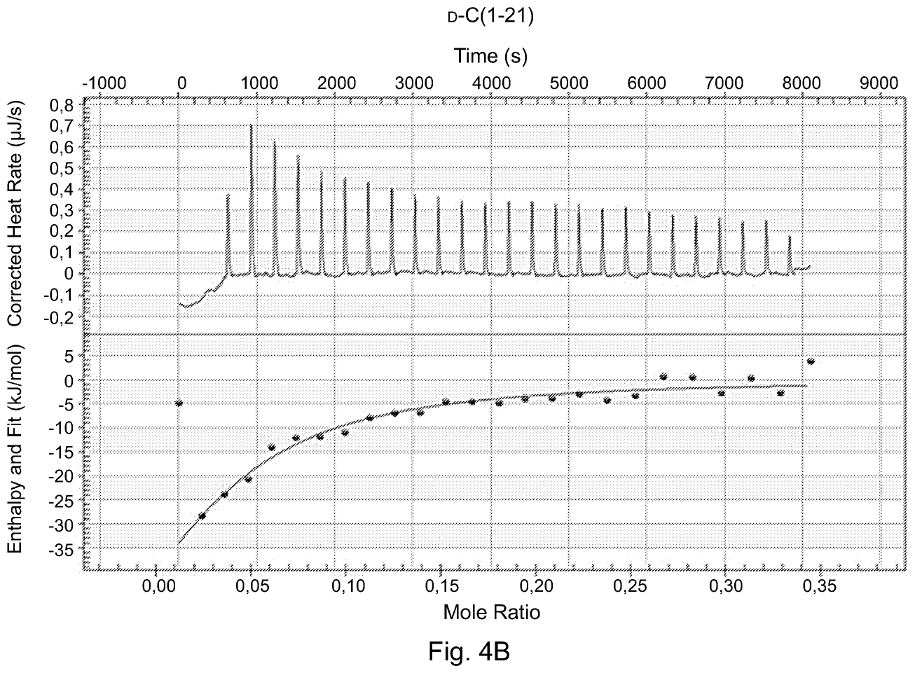

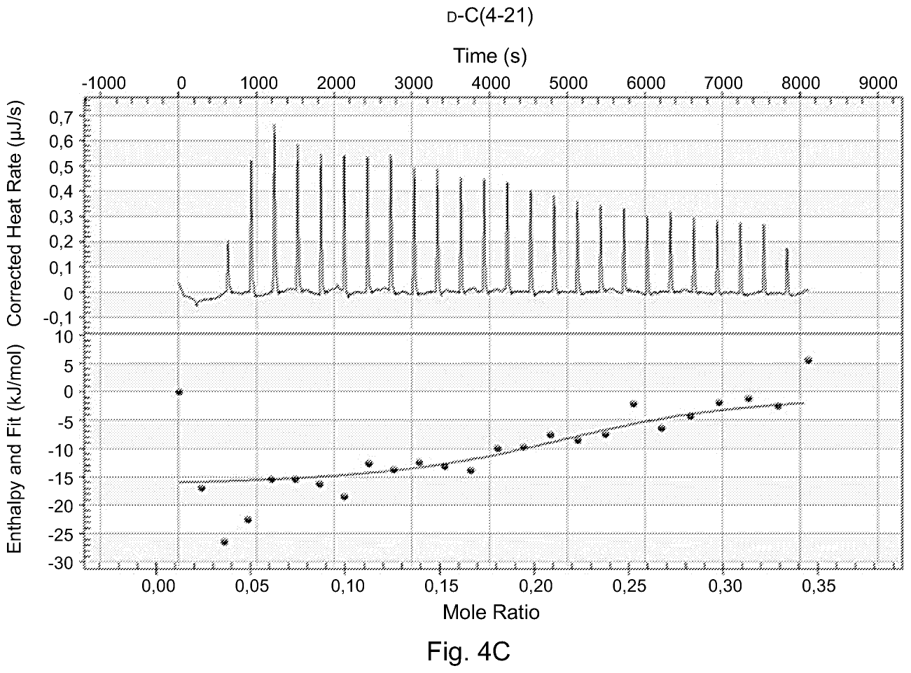

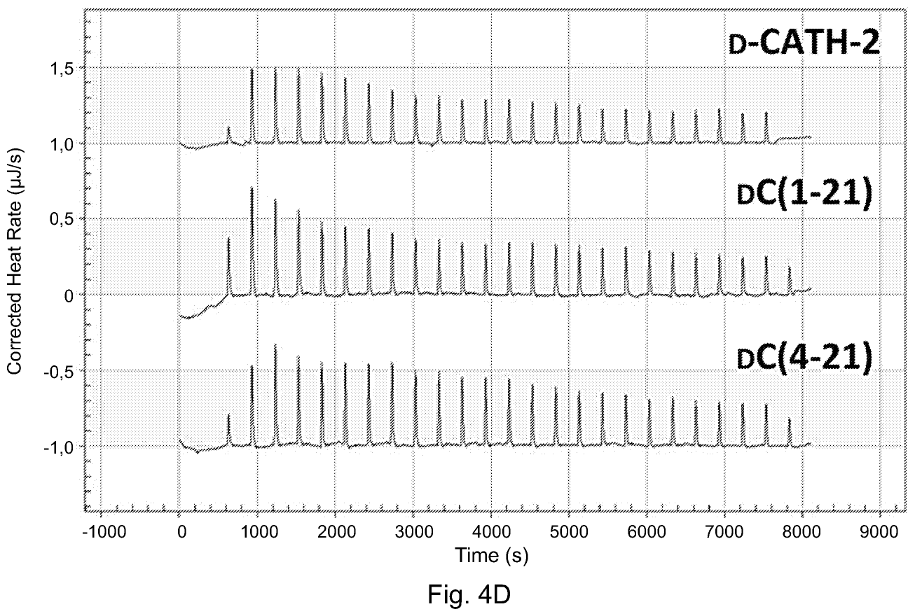

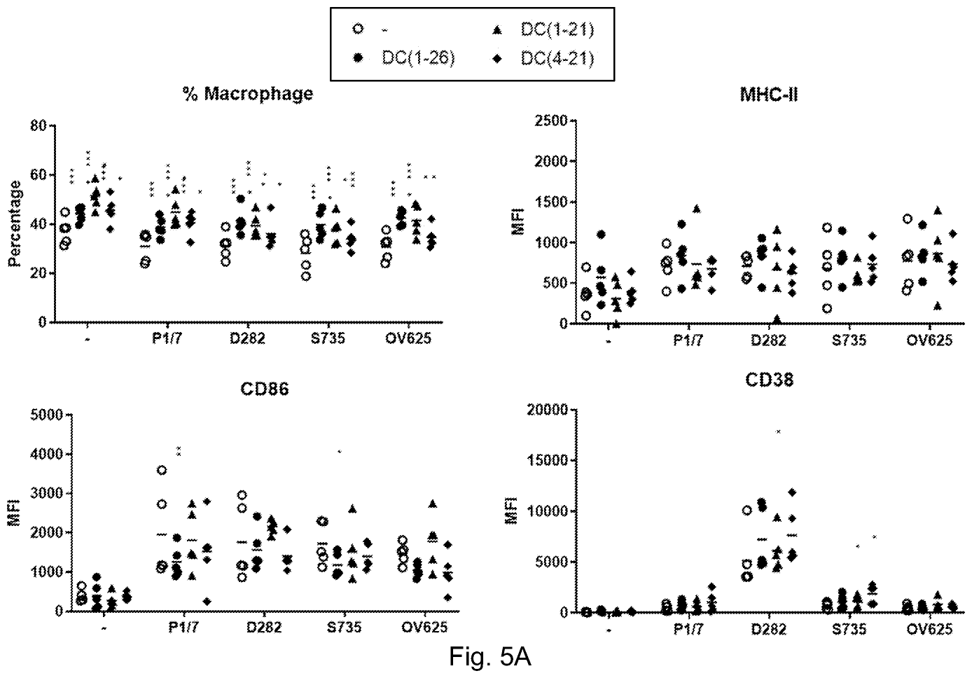

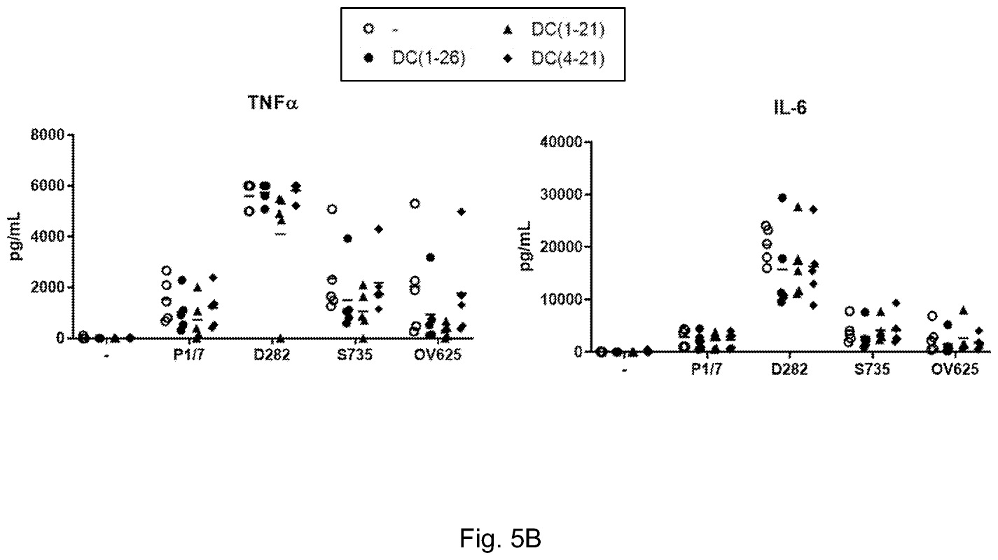

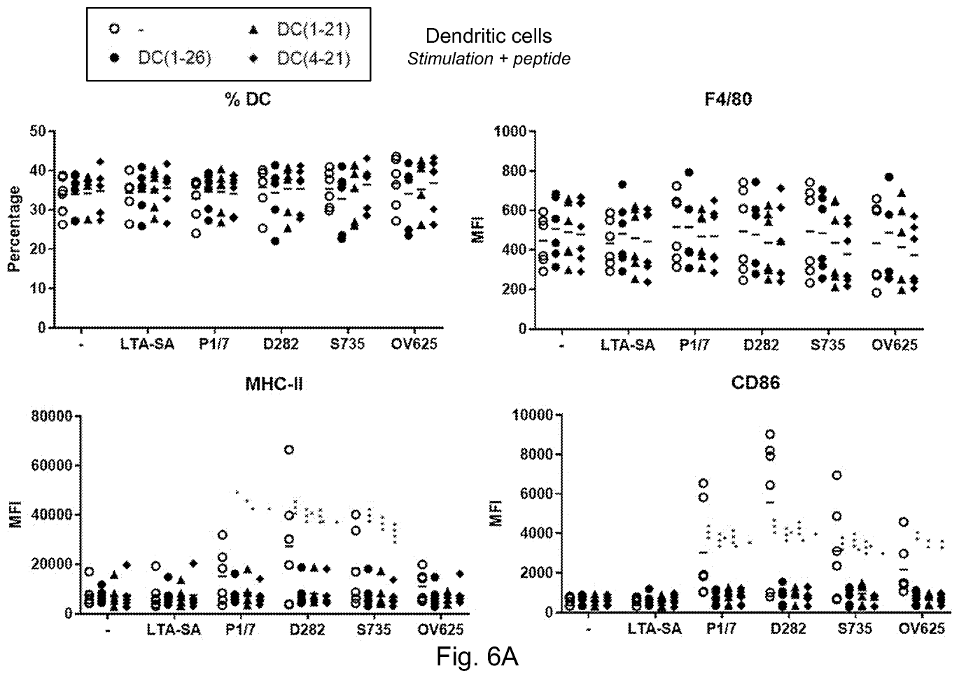

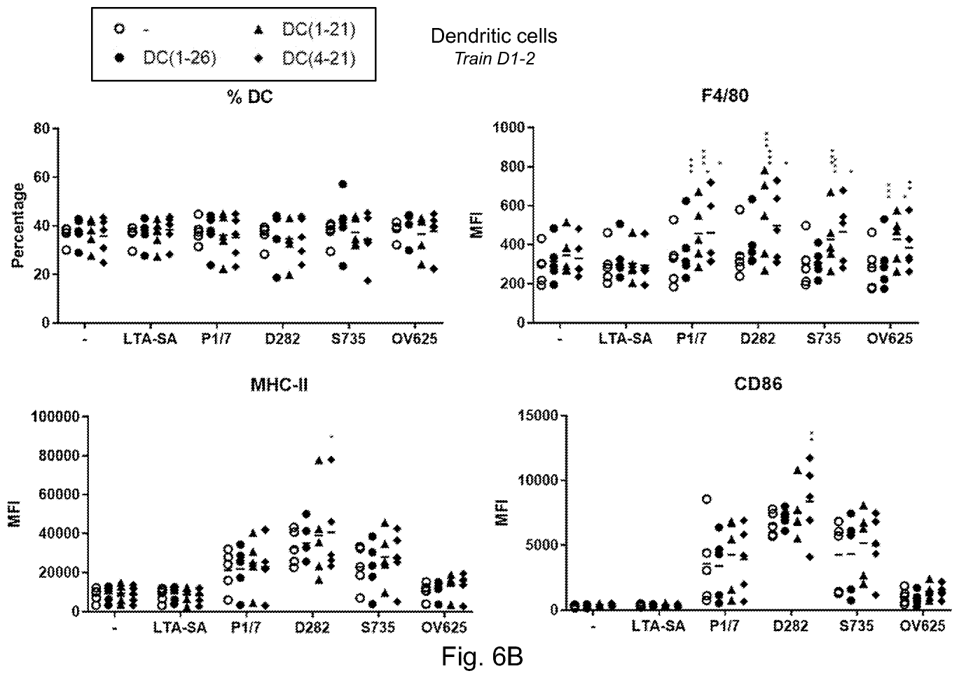

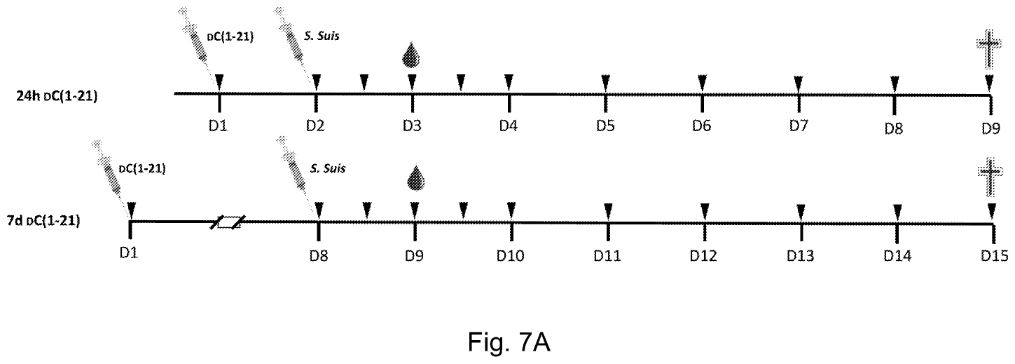

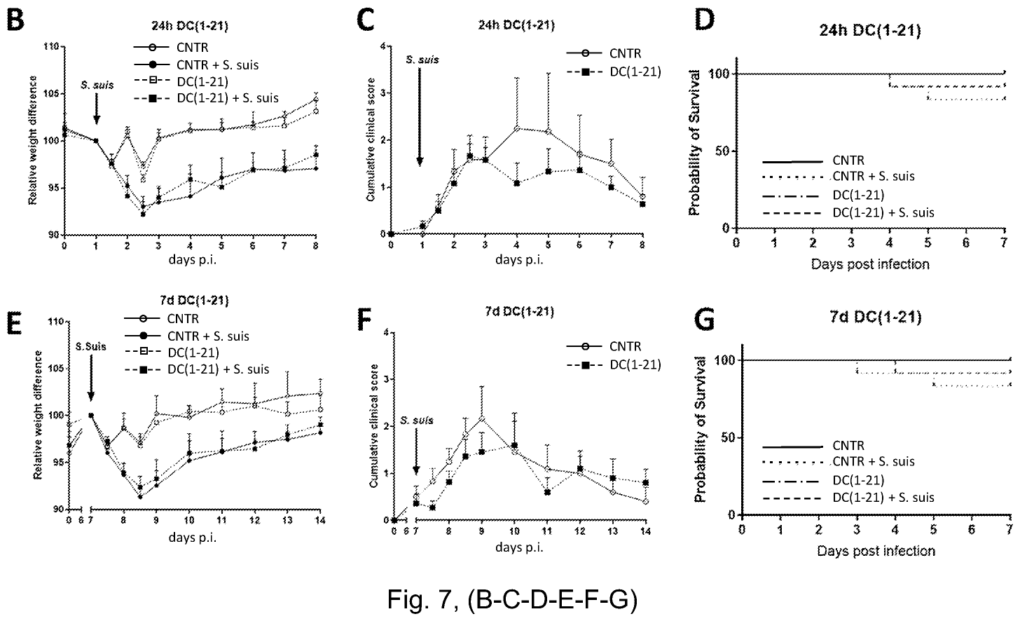

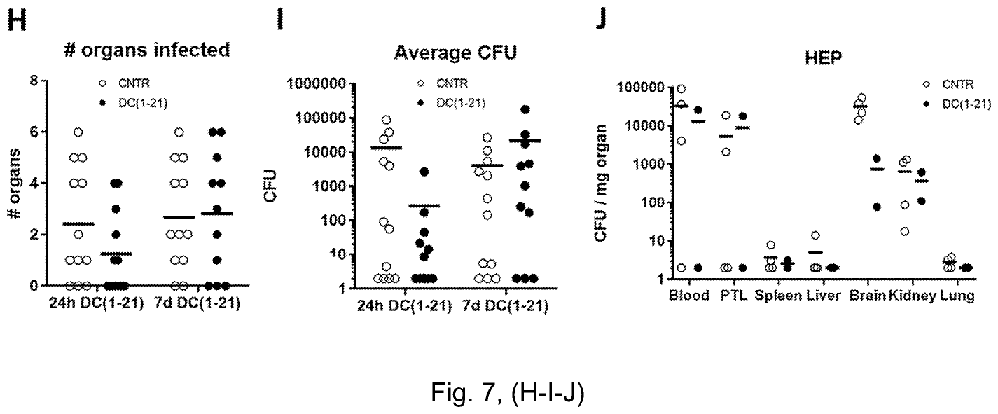

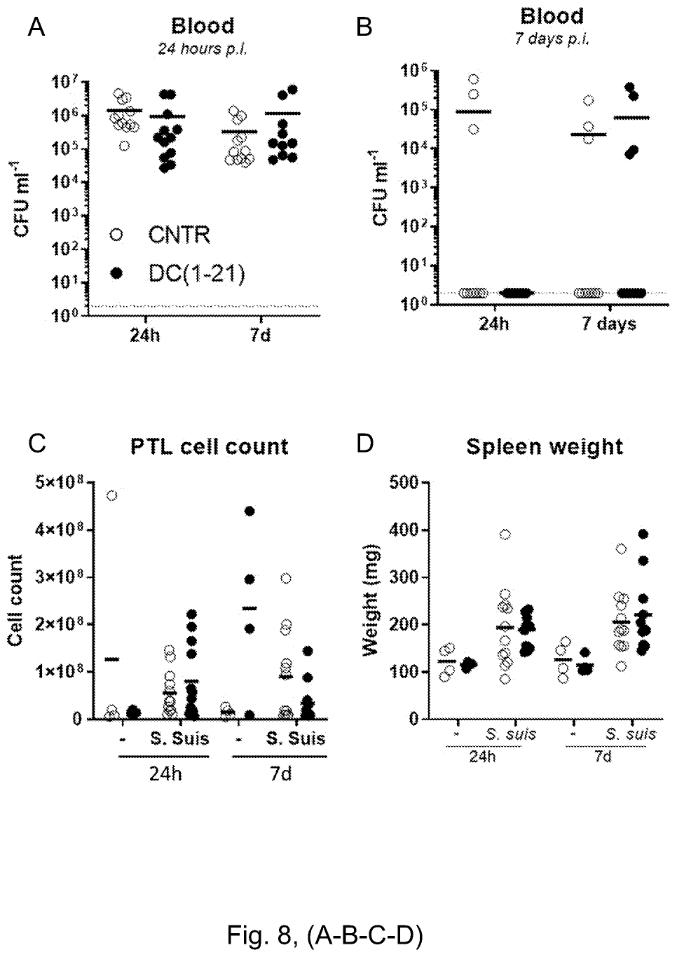

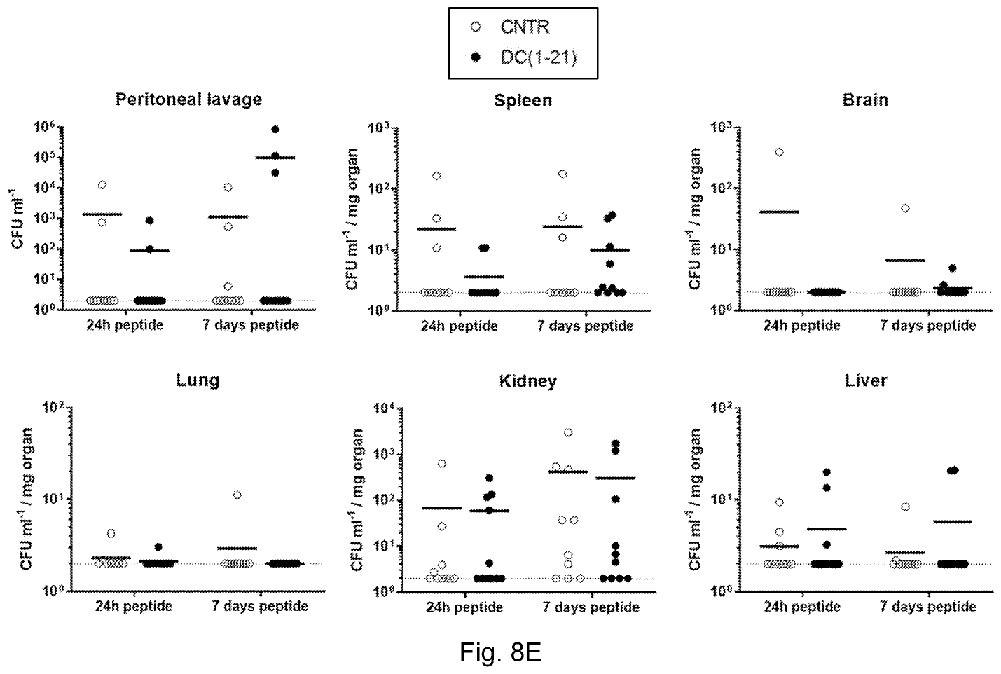

The biological form of CATH2 is known to inhibit LPS and LTA activation of a murine macrophage cell-line,[21] however, whether the full D antiomer of CATH2 is also capable of inhibiting LTA-induced of primary cultured murine BMDMs and BMDCs activation is unclear. In addition, cathelicidins can be cytotoxic to mammalian cells in higher concentrations.[19] Therefore, murine BMDMs and BMDCs were exposed to _{D}-CATH2, _{D}C(1-21) and _{D}C(4-21), either added at the end of the culture (day 6) or at the beginning of the culture (day 1) to observe any effects of the peptides on the cell viability and differentiation.
BMDMs were relative sensitive to addition of _{D}-CATH2 and its derivates, especially to _{D}C(1-21) (FIG. 2A), whereas BMDCs had some reduced viability, but starting from 2.5 μM peptides with no difference between the three (FIG. 2B). Both BMDM and BMDCs were less sensitive if the peptides were added at the beginning of the culture, with only a small reduction in viability at 5 μM (FIG. 2C and D). Analysis with flow cytometry also show a decrease in % BMDMs. The surviving BMDMs have an increased F4/80 expression and reduced MHC-II (FIG. 2E). A minor reduction in BMDCs is only visible at 5 μM, without affecting the other cell markers (FIG. 2F).
To analyze the effect of stimulation in combination with peptides, 1.25 μM was chosen as concentration on the border of cytotoxicity, but still influencing the expression of F4/80 with macrophages. Four different sub-strains of S. suis serotype 2 were mixed with 1.25 μM peptide and added to BMDMs at day 6. Activation of BMDMs in combination with the peptides, did not influence the percentage of macrophages as shown by flow cytometry. However, the upregulation of activation markers, like MHC-II, CD86 and CD38, was strongly inhibited by all three peptides for all four sub-strains (FIG. 1A). Similar results were found for BMDCs (FIG. 6A). In addition, the secretion of TNFa and IL-6 was inhibited in the presence of the peptides (FIG. 3B). To study the influence of the peptides on S. suis-induced activation in a more complex system, total splenocytes were activated ex vivo. In addition to whole live S. suis bacteria, pure LTA was used for activation. In the presence of peptides, nor LTA or whole S. suis bacteria were able to activate the splenocytes, shown by the inhibition of TNFα and IL-6 secretion (FIG. 3C).
To study whether to inhibitory effect on activation by the peptides to a direct interaction with LTA, the LTA binding capacity of peptides was tested using isothermal titration calorimetry (ITC). Although LTA- and S. suis-induced activation was strongly inhibited by all three peptides, is partially explained by its direct binding to LTA. With a dissociation coefficient K_{d} between 2-10 μM. Interestingly, _{D}C(1-21) bind less strong compared to the other two peptides, with a lower K_{d} and less peptide binding to one LTA molecule (FIG. 4 and Table 3), although the three peptides are equally efficient in inhibiting LTA- and S. suis-induced activation.

TABLE 3
ITC data

| | D-CATH2 | DC(1-21) | DC(4-21) |
|---|---|---|---|
| K_{d} (μM) | 3.039 | 10.22 | 2.12 |
| n | 0.543 | 0.207 | 1.209 |
| ΔH | −21.16 | −85.64 | −16.73 |
| (kJ/mol) | | | |
| ΔS | 37.39 | −180.6 | 54.68 |
| (J/mol · K) | | | |

Overview of ITC results of the binding capacity of 200 μM _{D}-CATH2, _{D}C(1-21) or _{D}-C(4-21) to 37.2 μM LTA-SA. K_{d}—dissociation coefficient (μM); n—number of peptide molecules binding to one LPS molecule; ΔH—enthalpy changes; -ΔS—entropy changes.
_{D}-CATH2 and its Derivatives Increases the BMDM Culture Efficiency To further study the effect of _{D}-CATH2 and its derivatives on macrophages, cells were exposed to the peptides 24 hours after the start of the culture for 24 hours. The efficiency of the BMDMs was enhanced by the early exposure of the peptides, shown by a higher percentage macrophages at day 6, which was most pronounced for _{D}C(1-21) (FIG. 5A). However, priming of the cells did not influence the activity of the cells for it did not change the activation markers MHC-II, CD86 and CD38 (FIG. 5A), nor was there any difference in cytokine expression by the peptide treated cells (FIG. 5B).
Similar results were found in the BMDC culture, exposing the cells to the peptides 24 hours after the start of the culture for 24 hours. Although the percentage of BMDCs at day 6 did not change, nor was there any difference in the expression of the activation markers, the macrophage marker F4/80 was increased, indicating a skewing towards macrophage like cells (FIG. 6B).

### _{D}C(1-21) Reduces the Clinical Symptoms of S. Suis P1/7 in Mice

Previously, our group has shown that in ovo injection of _{D}-CATH2 three days before hatch, protects the chickens up to 7 days post hatch for infection.[22] Since addition of _{D}-CATH2 and more specifically _{D}C(1-21) enhanced the efficiency of the murine BMDM culture and balanced the inflammatory response, we questioned whether injection of _{D}C(1-21) could boost the immune response in mice as well. Therefore, mice were injected with 1 mg/kg _{D}C(1-21) at day 1 subcutaneously and infected with 10⁷ CFU/ml S. suis P1/7 intraperitoneally 24 hours or 7 days post peptide injection. Mice were weighed twice a day during the acute phase of infection and daily until 7 days post infection (FIG. 7A). Both peptide-treated mice as control mice lost around 8% bodyweight up to 48 hours post infection and started to gain weight again due to S. suis infection, independent whether infection was started 24 hours (FIG. 7B) or 7 days post peptide injection (FIG. 7E). In addition, a cumulative clinical score was given twice a day during the acute phase of infection and daily during the chronic phase to the mice using a scoring table (Table 4). This shows a small reduction of cumulative clinical score for mice in the late stage of disease if mice were infected 24 hours post peptide injection (FIG. 7C) or at the acute phase of disease if infected 7 days post peptide injection (FIG. 7F). In addition to the reduced cumulative clinical score, treated mice had a higher change of survival when infection was given 24 hours (FIG. 7D) or 7 days post peptide injection (FIG. 7G).

Bacterial counts in the different organs were determined as well. 24 hours post infection, all mice, treated or not, had S. suis bacteria in the bloodstream between 10⁵-10⁶ CFU mL⁻¹ (FIG. 8A). After 7 days, most mice were able to deplete all bacteria in the blood, which was more efficient in 24 h _{D}C(1-21) treated mice compare to untreated (FIG. 8B). During the section at the end of the study, the peritoneum was washed and the cells present in the peritoneal lavage were counted, showing an increase upon infection, but not different for treated or untreated mice (FIG. 8C), nor were differences found with flow cytometry (data not shown), lastly, the amount of bacteria were determined showing that most mice were able to clear the bacteria in the peritoneum at similar rates in the treated and untreated groups (FIG. 8E). The spleens of S. suis infected mice were enlarged, showing an efficient infection model, however, no differences were found between treated and untreated mice (FIG. 8D). The amount of S. suis in the different organs was determined (FIG. 8E), only minor differences were found. However, counting the number of organs in which bacteria could be found, showed that 24 h _{D}C(1-21) treated mice had less positive organs (FIG. 7H) and less total CFU counts (FIG. 7I) compare to the untreated mice. This effect was not found for 7d _{D}C(1-21) treated mice. Also _{D}C(1-21) treated mice reaching the HEP before the end of the study, showed less bacterial counts, especially in the brain indicating a less severe course of disease(FIG. 7J). The immune cells were analyzed for the different organs; however, no differences were found between treated and untreated mice.

TABLE 4
Clinical scoring parameters for cumulative
scoring of S. suis-infected mice

| | SCORE | | |
|---|---|---|---|
| | 0 | 1 | 2 |
| BODY WEIGHT | Constant or gain | >5% weight | >20% weight |
| | | loss | loss |
| COAT | Flat and glossy | Rougher | Bloated |
| BREATHING | Rhythmic | Rapid | Rapid and |
| | | | abdominal |
| DEHYDRATION | Normal skin | Reduced skin | Persisting skin |
| | elasticity | elasticity | fold |
| BEARING | Normal | Curved back | Huddled |
| EYES | Normal | Moderately | Squeezed and |
| | | squeezed | swollen |
| ACTIVITY | Normal | Reduced activity | Apathy |
| LOCOMOTION | Normal | Reduced | Unsteady, |
| | | coordination | apraxia |

The cumulative clinical score was defined as the sum of the clinical scoring for eight parameters. Mice were euthanized for animal welfare reasons (humanized end point (HEP)) when they endured severe clinical signs (defined as: 2 days in a row a score of 2 on 3 of the 8 points) or in case of severe weight loss (>20%).

### References

1. Staats, J. J., Feder, I., Okwumabua, O. & Chengappa, M. M. Streptococcus suis: past and present. Vet Res Commun 21, 381-407, doi:10.1023/a:1005870317757 (1997).

2. Votsch, D., Willenborg, M., Weldearegay, Y. B. & Valentin-Weigand, P. Streptococcus suis—The “Two Faces” of a Pathobiont in the Porcine Respiratory Tract. Front Microbiol 9, 480, doi:10.3389/fmicb.2018.00480 (2018).

3. Kerdsin, A. et al. Fatal Septic Meningitis in Child Caused by Streptococcus suis Serotype 24. Emerg Infect Dis 22, 1519-1520, doi:10.3201/eid2208.160452 (2016).

4. Segura, M., Gottschalk, M. & Olivier, M. Encapsulated Streptococcus suis inhibits activation of signaling pathways involved in phagocytosis. Infect Immun 72, 5322-5330, doi:10.1128/IAI.72.9.5322-5330.2004 (2004).

5. Goyette-Desjardins, G., Auger, J. P., Xu, J., Segura, M. & Gottschalk, M. Streptococcus suis, an important pig pathogen and emerging zoonotic agent-an update on the worldwide distribution based on serotyping and sequence typing. Emerg Microbes Infect 3, e45, doi:10.1038/emi.2014.45 (2014).

6. van Dijk, A., et al., Identification of chicken cathelicidin-2 core elements involved in antibacterial and immunomodulatory activities. Mol Immunol, 2009. 46(13): p. 2465-73.

7. van Dijk, A., et al., Immunomodulatory and Anti-Inflammatory Activities of Chicken Cathelicidin-2 Derived Peptides. PLoS One, 2016. 11(2): p. e0147919.

8. Scheenstra, M.R., et al., Cathelicidins Modulate TLR-Activation and Inflammation. Front Immunol, 2020. 11: p. 1137.

9. Yang, D., et al., Alarmin-induced cell migration. Eur J Immunol, 2013. 43(6): p. 1412-8.

10. Coorens, M., et al., Interspecies cathelicidin comparison reveals divergence in antimicrobial activity, TLR modulation, chemokine induction and regulation of phagocytosis. Sci Rep, 2017. 7: p. 40874.

11. van Dijk, A., et al., Identification of chicken cathelicidin-2 core elements involved in antibacterial and immunomodulatory activities. Mol Immunol, 2009. 46(13): p. 2465-73.

12. van Dijk, A., et al., Immunomodulatory and Anti-Inflammatory Activities of Chicken Cathelicidin-2 Derived Peptides. PLoS One, 2016. 11(2): p. e0147919.

13. Mookherjee, N., et al., Antimicrobial host defence peptides: functions and clinical potential. Nat Rev Drug Discov, 2020. 19(5): p. 311-332.

14. Garton, M., et al., Method to generate highly stable D-amino acid analogs of bioactive helical peptides using a mirror image of the entire PDB. Proc Natl Acad Sci U S A, 2018. 115(7): p. 1505-1510.

15. Cuperus, T., et al., Protective effect of in ovo treatment with the chicken cathelicidin analog D-CATH-2 against avian pathogenic E. coli. Sci Rep, 2016. 6: p. 26622.

16. Schneider, V.A., et al., Prophylactic administration of chicken cathelicidin-2 boosts zebrafish embryonic innate immunity. Dev Comp Immunol, 2016. 60: p. 108-14.

17. de Greeff, A. et al. Genetic diversity of Streptococcus suis isolates as determined by comparative genome hybridization. BMC Microbiol 11, 161, doi:10.1186/1471-2180-11-161 (2011)

18. Seitz, M. et al. A novel intranasal mouse model for mucosal colonization by Streptococcus suis serotype 2. J Med Microbiol 61, 1311-1318, doi:10.1099/jmm.0.043885-0 (2012).

19. Scheenstra, M. R. et al. Cathelicidins PMAP-36, LL-37 and CATH-2 are similar peptides with different modes of action. Sci Rep 9, 4780, doi:10.1038/s41598-019-41246-6 (2019).

20. Veldhuizen, E. J. A. et al. Antimicrobial and Immunomodulatory Activity of PMAP-23 Derived Peptides. Protein Pept Lett 24, 609-616, doi:10.2174/0929866524666170428150925 (2017).

21. Coorens, M., Scheenstra, M. R., Veldhuizen, E. J. & Haagsman, H. P. Interspecies cathelicidin comparison reveals divergence in antimicrobial activity, TLR modulation, chemokine induction and regulation of phagocytosis. Sci Rep 7, 40874, doi:10.1038/srep40874 (2017).

22. Cuperus, T., van Dijk, A., Matthijs, M. G., Veldhuizen, E. J. & Haagsman, H. P. Protective effect of in ovo treatment with the chicken cathelicidin analog D-CATH-2 against avian pathogenic E. coli. Sci Rep 6, 26622, doi:10.1038/srep26622 (2016).

1. A method for the treatment or prevention of a Streptococcus suis (S. suis) infection in a subject in need thereof, comprising administering CATH2 or a derivative thereof to the subject.

2. (canceled)

3. (canceled)

4. (canceled)

5. The method according to claim 1 wherein said S. suis is S. suis serotype 2, serotype 9, serotype 1 or serotype 3.

6. The method according to claim 1 wherein said S. suis is serotype 2.

7. The method according to claim 1 wherein the subject in need thereof is suffering from a S. suis infection or at risk of suffering from a S. suis infection.

8. The method according to claim 1 comprising administering said CATH2 or derivative thereof to subjects of a population of subjects wherein a S. suis infection has been established in one or more subjects of said population.

9. The method according to claim 1 wherein the subject is administered the CATH2 derivative twice.

10. The method according to claim 9 wherein the subject is administered the CATH2 derivative with an interval of at least 2 days.

11. The method according to claim 1 wherein the subject is poultry and the administration is performed in ovo and/or after hatch.

12. The method according to claim 1 comprising inducing or promoting innate immune memory in the subject.

13. The method according to claim 1 comprising improving or enhancing antimicrobial treatment with an antimicrobial agent.

14. A method for inhibiting Streptococcus suis comprising administering CATH2 or a derivative thereof to the S. suis.

15. (canceled)

16. The method according to claim 1 wherein the CATH2 derivative is selected from the group consisting of DCATH2, a C-terminally and/or N-terminally truncated CATH2 and a C-terminally or N-terminally truncated DCATH2.

17. The method according to claim 16, wherein the CATH2 derivative is selected from the group consisting of DCATH2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

18. The method according to claim 16, wherein the CATH2 or derivative is DCATH2, DCATH2(1-21) or DCATH2(4-21).

19. The method according to claim 1 wherein the CATH2 or a derivative thereof is combined with an adjuvant specific for innate immunity.

20. The method according to claim 1 wherein the CATH2 or derivative thereof is administered before, after or simultaneously with a treatment with a S. suis or an antigenic part thereof.

21. The method according to claim 7 wherein the subject suffering from a S. suis infection or at risk of suffering from a S. suis infection is a subject that is in contact with subjects suffering from said infection.

22. The method according to claim 19 wherein the adjuvant specific for innate immunity is selected from the group consisting of a toll-like receptor (TLR) ligand, β-glucan, muramyl dipeptide (MDP), Bacille Calmette-Guerin (BCG), CpG oligodeoxynucleotide.
